(19) United States
(12) Patent Application Publication
(10) Pub. No.: US 2021/0180094 A1
(43) Pub. Date: Jun. 17, 2021

(54) Method of Producing Lipid

(71) Applicant: KAO CORPORATION, Tokyo (JP)

(72) Inventor: Akihito KAWAHARA, Wakayama-shi (JP)

(73) Assignee: Kao Corporation, Tokyo (JP)

(21) Appl. No.: 17/046,867

(22) PCT Filed: Apr. 9, 2019
(86) PCT No.: PCT/JP2019/015521

§ 371 Date: Oct. 12, 2020

Related U.S. Application Data
(60) Provisional application No. 62/659,793, filed on Apr. 19, 2018.

(51) Int. Cl. C12P 7/64; C12N 9/04; C12N 9/16; C12N 15/70

(52) U.S. Cl. CPC C12P 7/6409; C12Y 101/01212; C12N 15/70; C12N 9/16; C12Y 301/02014; C12N 9/0006

(57) ABSTRACT

A method of producing lipids, containing the steps of:
culturing a transformant wherein a gene encoding the following protein (A) or (B), and a gene encoding the following protein (C) or (D) are introduced into a host cell, and
producing medium-chain fatty acids or the lipids containing the same as components:
   (A) a protein consisting of the amino acid sequence set forth in SEQ ID NO: 2;
   (B) a protein consisting of an amino acid sequence having 60% or more identity with the amino acid sequence set forth in SEQ ID NO: 2, and having acyl-ACP thioesterase activity;
   (C) a protein consisting of the amino acid sequence set forth in SEQ ID NO: 4; and
   (D) a protein consisting of an amino acid sequence having 60% or more identity with the amino acid sequence set forth in SEQ ID NO: 4, and having β-ketoacyl-ACP reductase activity.

### TECHNICAL FIELD

The present invention relates to a method of producing a lipid.

Further, the present invention relates to a transformant for use in this method.

### BACKGROUND ART

Fatty acids are one of the principal components of lipids. In vivo, fatty acids are bonded to glycerin via an ester bond to form lipids (fat and oil) such as triacylglycerol. Further, many animals and plants also store and utilize fatty acids as an energy source. These fatty acids and lipids stored in animals and plants are widely utilized for food or industrial use.

For example, higher alcohol derivatives that are obtained by reducing higher fatty acids having approximately 12 to 18 carbon atoms are used as surfactants. Alkyl sulfuric acid ester salts, alkylbenzenesulfonic acid salts and the like are utilized as anionic surfactants. Further, polyoxyalkylene alkyl ethers, alkyl polyglycosides and the like are utilized as nonionic surfactants. These surfactants are used for detergents, disinfectants, or the like. Cationic surfactants such as alkylamine salts and mono- or dialkyl-quaternary amine salts, as other higher alcohol derivatives, are commonly used for fiber treatment agents, hair conditioning agents, disinfectants, or the like. Further, benzalkonium type quaternary ammonium salts are commonly used for disinfectants, antiseptics, or the like. Furthermore, fats and oils derived from plants are also used as raw materials of biodiesel fuels.

Moreover, a medium-chain fatty acid having 8 or 10 carbon atoms is used for health food or an etching agent. Moreover, alcohol derivatives that are obtained by reducing the medium-chain fatty acid having 8 or 10 carbon atoms are also used as industrial raw materials for products such as cosmetics, surfactants and plasticizers.

Fatty acids and lipids are widely used for various applications shown above. Therefore, it has been attempted to enhance productivity of fatty acids or lipids in vivo by using plants and the like. Furthermore, the applications and usefulness of fatty acids depend on the number of carbon atoms therein. Therefore, controlling of the number of carbon atoms of the fatty acids, namely, a chain length thereof has also been attempted. Furthermore, attention has been paid to a method of producing biochemicals including fatty acids by culturing a microorganism such as Escherichia coli using a renewable energy source such as sunlight and biomass.

For example, it is known that productivity of medium-chain fatty acids having 8 or 10 carbon atoms in a transformant obtained is improved by introducing a gene encoding an acyl-ACP (acyl-carrier protein) thioesterase (hereinafter, also merely referred to as “TE”) derived from plants belonging to the genus Cuphea, such as Cuphea palustris and Cuphea hookeriana or a variant thereof into a host (see Patent Literatures 1 and 2).

### CITATION LIST

### Patent Literatures

Patent Literature 1: U.S. Pat. No. 5,955,329 A

Patent Literature 2: US 2011/0020883 A1

### SUMMARY OF INVENTION

The present invention relates to a method of producing lipids, containing the steps of:

culturing a transformant wherein a gene encoding the following protein (A) or (B), and a gene encoding the following protein (C) or (D) are introduced into a host cell, and

producing medium-chain fatty acids or lipids containing the same as components:

(A) a protein consisting of the amino acid sequence set forth in SEQ ID NO: 2;
(B) a protein consisting of an amino acid sequence having 60% or more identity with the amino acid sequence set forth in SEQ ID NO: 2, and having acyl-ACP thioesterase activity;
(C) a protein consisting of the amino acid sequence set forth in SEQ ID NO: 4; and
(D) a protein consisting of an amino acid sequence having 60% or more identity with the amino acid sequence set forth in SEQ ID NO: 4, and having β-ketoacyl-ACP reductase activity.

Further, the present invention relates to a transformant, wherein expression of a gene encoding the protein (A) or (B), and a gene encoding the protein (C) or (D) is enhanced in a host cell.

### DESCRIPTION OF EMBODIMENTS

The present invention relates to a method of producing lipids, which improves productivity of medium-chain fatty acids or the lipids containing the same as components.

Further, the present invention relates to a transformant in which productivity of medium-chain fatty acids or lipids containing the same as components is improved, and which can be preferably used in the method.

As reported in Patent Literatures 1 and 2, for production of medium-chain fatty acids using a transformant of Escherichia coli, cyanobacteria or the like into which a gene encoding TE derived from a plant belonging to the genus Cuphea has been introduced, attempts have been also made to improve amount of fatty acids production.

In the first step of fatty acid synthesis, an acetoacetyl ACP is produced by condensation reaction of an acetyl-ACP (or acetyl-CoA) and a malonyl ACP. Then, a keto group of the acetoacetyl ACP is reduced by a β-ketoacyl-ACP reductase (hereinafter, also referred to as “FabG”) to produce a hydroxybutyryl ACP. Subsequently, a β-hydroxyacyl-ACP dehydrase dehydrates the hydroxybutyryl ACP to produce a crotonyl ACP. Finally, the crotonyl ACP is reduced by an enoyl-ACP reductase (hereinafter, also referred to as “Fabl”) to produce a butyryl ACP. Such a series of reaction is used to produce a butyryl ACP by adding two carbon atoms to the carbon chain of the acyl group from an acetyl-ACP. Subsequently, the same reaction is repeated to extend the carbon chain of an acyl-ACP.

Among the above-mentioned enzymes involved in fatty acid synthesis, Fabl of Escherichia coli utilizes NADPH and NADH as coenzymes in catalyzing the above reaction. By contrast, FabG of Escherichia coli is known to utilize just NADPH in catalyzing the above reaction. Here, proper balance of intracellular coenzymes is reportedly important in fatty acid synthesis (see Bergler et al., Eur. J. Biochem., 1996. 242, p. 689-694; and Toomey R E and Wakil S J, Biochim. Biophys. Acta., 1966. 116, p. 189-197).

Therefore, with regard to a microorganism prepared by introducing a gene encoding TE derived from a plant belonging to the genus Cuphea, the present inventors sought to optimally improve productivity of medium-chain fatty acids by focusing on a type of FabG that uses NADH as a coenzyme. For example, as mentioned above, FabG of Escherichia coli is known to use only NADPH as a coenzyme. The inventors therefore came to the conclusion that a host having a type of FabG that utilizes only NADPH as a coenzyme (hereinafter, also referred to as “NADPH-type FabG”) might be transformable with a type of FabG that utilizes NADH as a coenzyme (hereinafter, also referred to as “NADH-type FabG”). In this case, both NADH and NADPH would be utilized for fatty acid synthesis without incurring any energetic competition. This might result in an increase in productivity of medium-chain fatty acids. The present inventor conducted further research based on this thinking. As a result, the present inventor discovered that productivity of medium-chain fatty acids is further improved by introducing a gene encoding NADH-type FabG to enhance expression of NADH-type FabG into a host containing a gene encoding TE derived from a plant belonging to the genus Cuphea.

The present invention was completed based on these findings.

According to the method of producing lipids of the present invention, productivity of medium-chain fatty acids or the lipids containing the same as components can be improved.

Moreover, the transformant of the present invention is excellent in productivity of medium-chain fatty acids or lipids containing the same as components.

Other and further features and advantages of the invention will appear more fully from the following description.

The term “lipid(s)” in the present specification, covers a simple lipid such as a neutral lipid (monoacylglycerol (MAG), diacylglycerol (DAG), triacylglycerol (TAG), or the like), wax, and a ceramide; a complex lipid such as a phospholipid, a glycolipid, and a sulfolipid; and a derived lipid obtained from the lipid such as a fatty acid (free fatty acid), alcohols, and hydrocarbons.

The fatty acids categorized into the derived lipid generally refer to the fatty acids per se and mean “free fatty acids”. In the present invention, the fatty acid group or the acyl group in molecules of a simple lipid and a complex lipid is expressed as “fatty acid residue”. Then, unless otherwise specified, a term “fatty acid” is used as a generic term for “free fatty acid” and “fatty acid residue”.

Moreover, a term “fatty acids or lipids containing the same as components” in the present specification is generically used including “free fatty acids” and “lipids having the fatty acid residues”. Further, a term “fatty acid composition” in the present specification means a weight proportion of each fatty acid relative to the weight of whole fatty acids (total fatty acids) obtained by totaling the free fatty acids and the fatty acid residues described above regarding as fatty acids. The weight (production amount) of the fatty acids or the fatty acid composition can be measured according to the method used in Examples.

In the present specification, the description of “Cx:y” for the fatty acid or the acyl group constituting the fatty acid means that the number of carbon atoms is “x” and the number of double bonds is “y”. The description of “Cx” means a fatty acid or an acyl group having “x” as the number of carbon atoms. In the present specification, the identity of the nucleotide sequence and the amino acid sequence is calculated through the Lipman-Pearson method (Science, 1985, vol. 227, p. 1435-1441). Specifically, the identity can be determined through use of a homology analysis (search homology) program of genetic information processing software Genetyx-Win with Unit size to compare (ktup) being set to 2.

It should be note that, in the present specification, the “stringent conditions” includes, for example, the method described in Molecular Cloning—A LABORATORY MANUAL THIRD EDITION [Joseph Sambrook and David W. Russell, Cold Spring Harbor Laboratory Press], and examples thereof include conditions where hybridization is performed by incubating a solution containing 6×SSC (composition of 1×SSC: 0.15 M sodium chloride, 0.015 M sodium citrate, pH 7.0), 0.5% SDS, 5×Denhardt's solution and 100 mg/mL herring sperm DNA together with a probe at 65° C. for 8 to 16 hours.

Furthermore, in the present specification, the term “upstream” of a gene means a region subsequent to a 5′ side of a targeted gene or region, and not a position from a translational initiation site. On the other hand, the term “downstream” of the gene means a region subsequent to a 3′ side of the targeted gene or region.

Note that, in the present specification, the term “medium-chain” means that the number of carbon atoms of the acyl group is 8 or more and less than 10, preferably 8 or 10, more preferably 8. Further, productivity of fatty acid and lipid in a transformant can be measured by a method used in Examples.

TE is an enzyme involved in the biosynthesis pathway of fatty acids and derivatives thereof (such as triacylglycerol (triglyceride)). This enzyme hydrolyzes a thioester bond of an acyl-ACP (a composite composed of an acyl group as a fatty acid residue and an acyl carrier protein), which is an intermediate in the process of fatty acid biosynthesis, to form free fatty acids in a plastid such as a chloroplast of plants and algae or in a cytoplasm of bacteria, fungi and animals. The function of the TE terminates the fatty acid synthesis on the ACP, and then the thus-hydrolyzed fatty acid is supplied to the synthesis of triacylglycerol or the like. Several TEs having different reaction specificities depending on the number of carbon atoms and the number of unsaturated bonds of the acyl group (fatty acid residue) constituting the acyl-ACP substrate are identified, and TE is considered to be an important factor in determining the fatty acid composition of an organism.

In the present specification, the term acyl-ACP thioesterase activity (hereinafter, also referred to as “TE activity”) means an activity of hydrolyzing the thioester bond of the acyl-ACP.

In the present invention, the protein (A) or (B) is used as a TE.

The protein (A) consisting of the amino acid sequence set forth in SEQ ID NO: 2 is a part of an amino acid sequence of a wild-type TE derived from Cuphea palustris which consists of the amino acid sequence set forth in SEQ ID NO: 17. In the amino acid sequence set forth in SEQ ID NO: 2, region of putative signal sequence (amino acid sequence at positions 2 to 57 of SEQ ID NO: 17) is deleted from the full length of amino acid sequence of the wild-type TE. That is, in the amino acid sequence set forth in SEQ ID NO: 2, amino acids at positions 1 to 57 are removed from the amino acid sequence set forth in SEQ ID NO: 17, and a protein synthesis initiation amino acid (methionine) is added to N-terminal side of amino acid at position 58. It is known that the region of the 58th to 411st positions in the amino acid sequence of a wild-type TE derived from Cuphea palustris is an important and sufficient region for exhibiting the TE activity. That is, the protein consisting of the amino acid sequence set forth in SEQ ID NO: 2 has the TE activity and acts as TE, because the protein has the sufficient region for the TE activity. A TE derived from plants belonging to the genus Cuphea has high specificity to a medium-chain acyl-ACP having 8 or 10 carbon atoms, and thereby the TE is suitably used for improvement of productivity of medium-chain fatty acids having 8 or 10 carbon atoms in a transformant.

Hereinafter, the protein (A) is also referred to as “CpTE”.

The protein (B) consists of an amino acid sequence having 60% or more identity with the amino acid sequence set forth in SEQ ID NO: 2, and has TE activity. In general, it is known that an amino acid sequence encoding an enzyme protein does not necessarily exhibit enzyme activity unless the sequence in the whole region is conserved, and there exists a region in which the enzyme activity is not influenced even if the amino acid sequence is changed. In such a region which is not essential to the enzyme activity, even if the mutation of the amino acid, such as deletion, substitution, insertion and addition thereof is introduced thereinto, the activity inherent to the enzyme can be maintained. Also in the present invention, such a protein can be used in which the TE activity is kept and a part of the amino acid sequence is subjected to mutation.

In the protein (B), the identity with the amino acid sequence set forth in SEQ ID NO: 2 is 60% or more, preferably 65% or more, more preferably 70% or more, further preferably 75% or more, further preferably 80% or more, further preferably 85% or more, further preferably 90% or more, further preferably 93% or more, further preferably 95% or more, further preferably 96% or more, further preferably 97% or more, further preferably 98% or more, and furthermore preferably 99% or more, in view of TE activity.

Further, specific examples of the protein (B) include a protein in which 1 or several (for example 1 or more and 142 or less, preferably 1 or more and 124 or less, more preferably 1 or more and 106 or less, further preferably 1 or more and 88 or less, furthermore preferably 1 or more and 71 or less, furthermore preferably 1 or more and 53 or less, furthermore preferably 1 or more and 35 or less, furthermore preferably 1 or more and 24 or less, furthermore preferably 1 or more and 17 or less, furthermore preferably 1 or more and 14 or less, furthermore preferably 1 or more and 10 or less, furthermore preferably 1 or more and 7 or less, and furthermore preferably 1 or more and 3 or less) amino acids are deleted, substituted, inserted or added to the amino acid sequence set forth in SEQ ID NO: 2, and having TE activity.

Specific examples of the protein (B) that is preferably used in the present invention include a protein in which an amino acid at a specific position in the amino acid sequence set forth in SEQ ID NO: 2 is substituted (hereinafter, also referred to as “CpTE variant”), and a protein which contains the amino acid substitution. Specificity to a medium-chain acyl-ACP is improved in the CpTE variant as compared with the protein (A). That is, in comparison with the wild type CpTE, the CpTE variant selectively utilizes a medium-chain acyl-ACP as a substrate and has improved activity of hydrolyzing this substrate.

From viewpoints of improving specificity to the medium-chain acyl-ACP, and improving productivity of medium-chain fatty acids or lipids containing the same as components, the amino acid sequence of the protein (B) preferably has at least one amino acid substitution selected from the group consisting of the following (B-1) to (B-11):

(B-1) substitution of isoleucine for an amino acid at a position 257 of the amino acid sequence set forth in SEQ ID NO: 2 or at a position corresponding thereto (preferably or generally, leucine);
(B-2) substitution of arginine for an amino acid at a position 251 of the amino acid sequence set forth in SEQ ID NO: 2 or at a position corresponding thereto (preferably or generally, threonine);
(B-3) substitution of lysine for an amino acid at a position 251 of the amino acid sequence set forth in SEQ ID NO: 2 or at a position corresponding thereto (preferably or generally, threonine);
(B-4) substitution of histidine for an amino acid at a position 251 of the amino acid sequence set forth in SEQ ID NO: 2 or at a position corresponding thereto (preferably or generally, threonine);
(B-5) substitution of isoleucine for an amino acid at a position 254 of the amino acid sequence set forth in SEQ ID NO: 2 or at a position corresponding thereto (preferably or generally, tryptophan);
(B-6) substitution of tyrosine for an amino acid at a position 254 of the amino acid sequence set forth in SEQ ID NO: 2 or at a position corresponding thereto (preferably or generally, tryptophan);
(B-7) substitution of methionine for an amino acid at a position 257 of the amino acid sequence set forth in SEQ ID NO: 2 or at a position corresponding thereto (preferably or generally, leucine);
(B-8) substitution of valine for an amino acid at a position 257 of the amino acid sequence set forth in SEQ ID NO: 2 or at a position corresponding thereto (preferably or generally, leucine);
(B-9) substitution of phenylalanine for an amino acid at a position 257 of the amino acid sequence set forth in SEQ ID NO: 2 or at a position corresponding thereto (preferably or generally, leucine);
(B-10) substitution of cysteine for an amino acid at a position 266 of the amino acid sequence set forth in SEQ ID NO: 2 or at a position corresponding thereto (preferably or generally, valine); and
(B-11) substitution of tyrosine for an amino acid at a position 271 of the amino acid sequence set forth in SEQ ID NO: 2 or at a position corresponding thereto (preferably or generally, tryptophan).

The “position corresponding thereto” in the amino acid sequence or the nucleotide sequence can be determined by comparing an objective amino acid sequence with a reference sequence to align (provide alignment to) the sequence so as to give the maximum homology for a conserved amino acid residue existing in each amino acid sequence. The alignment can be executed by using a publicly known algorithm, and the procedures are publicly known to a person skilled in the art. The alignment can be manually performed, for example, based on the Lipman-Pearson method mentioned above; or alternatively, can be performed by using the Clustal W multiple alignment program (Nucleic Acids Res., 1994, vol. 22, p. 4673-4680) by default. The Clustal W is available from websites: for example, European Bioinformatics Institute: EBI, (www.ebi.ac.uk/index.html) and DNA Data Bank of Japan (DDBJ, [www.ddbj.nig.ac.jplWelcome-j.html]) managed by the National Institute of Genetics.

The protein (B) preferably has at least one amino acid substitution selected from the group consisting of the following (B-12) to (B-19), in addition to at least one amino acid substitution selected from the group consisting of the (B-1) to (B-11). In a case having these amino acid substitutions, specificity of the medium-chain acyl-ACP and productivity of medium-chain fatty acids or lipids containing the same as components are further improved.

(B-12) substitution of isoleucine for an amino acid at a position 106 of the amino acid sequence set forth in SEQ ID NO: 2 or at a position corresponding thereto (preferably or generally, valine);
(B-13) substitution of lysine for an amino acid at a position 108 of the amino acid sequence set forth in SEQ ID NO: 2 or at a position corresponding thereto (preferably or generally, asparagine);
(B-14) substitution of arginine for an amino acid at a position 108 of the amino acid sequence set forth in SEQ ID NO: 2 or at a position corresponding thereto (preferably or generally, asparagine);
(B-15) substitution of isoleucine for an amino acid at a position 110 of the amino acid sequence set forth in SEQ ID NO: 2 or at a position corresponding thereto (preferably or generally, valine);
(B-16) substitution of methionine for an amino acid at a position 110 of the amino acid sequence set forth in SEQ ID NO: 2 or at a position corresponding thereto (preferably or generally, valine);
(B-17) substitution of leucine for an amino acid at a position 110 of the amino acid sequence set forth in SEQ ID NO: 2 or at a position corresponding thereto (preferably or generally, valine);
(B-18) substitution of phenylalanine for an amino acid at a position 110 of the amino acid sequence set forth in SEQ ID NO: 2 or at a position corresponding thereto (preferably or generally, valine); and
(B-19) substitution of isoleucine for an amino acid at a position 118 of the amino acid sequence set forth in SEQ ID NO: 2 or at a position corresponding thereto (preferably or generally, cysteine).

In the present invention, the protein (B) preferably has the amino acid substitution of (B-1) and at least one amino acid substitution selected from the group consisting of the (B-12) to (B-19). Namely, in the present invention, the protein (B) more preferably has amino acid substitutions selected from the group consisting of the following (B-1_B-12) to (B-1_B-19).

(B-1_B-12) substitutions of isoleucine for an amino acid at a position 257 of the amino acid sequence set forth in SEQ ID NO: 2 or at a position corresponding thereto (preferably or generally, leucine), and isoleucine for an amino acid at a position 106 of the amino acid sequence set forth in SEQ ID NO: 2 or at a position corresponding thereto (preferably or generally, valine);
(B-1_B-13) substitutions of isoleucine for an amino acid at a position 257 of the amino acid sequence set forth in SEQ ID NO: 2 or at a position corresponding thereto (preferably or generally, leucine), and lysine for an amino acid at a position 108 of the amino acid sequence set forth in SEQ ID NO: 2 or at a position corresponding thereto (preferably or generally, asparagine);
(B-1_B-14) substitutions of isoleucine for an amino acid at a position 257 of the amino acid sequence set forth in SEQ ID NO: 2 or at a position corresponding thereto (preferably or generally, leucine), and arginine for an amino acid at a position 108 of the amino acid sequence set forth in SEQ ID NO: 2 or at a position corresponding thereto (preferably or generally, asparagine);
(B-1_B-15) substitutions of isoleucine for an amino acid at a position 257 of the amino acid sequence set forth in SEQ ID NO: 2 or at a position corresponding thereto (preferably or generally, leucine), and isoleucine for an amino acid at a position 110 of the amino acid sequence set forth in SEQ ID NO: 2 or at a position corresponding thereto (preferably or generally, valine);
(B-1_B-16) substitutions of isoleucine for an amino acid at a position 257 of the amino acid sequence set forth in SEQ ID NO: 2 or at a position corresponding thereto (preferably or generally, leucine), and methionine for an amino acid at a position 110 of the amino acid sequence set forth in SEQ ID NO: 2 or at a position corresponding thereto (preferably or generally, valine);
(B-1_B-17) substitutions of isoleucine for an amino acid at a position 257 of the amino acid sequence set forth in SEQ ID NO: 2 or at a position corresponding thereto (preferably or generally, leucine), and leucine for an amino acid at a position 110 of the amino acid sequence set forth in SEQ ID NO: 2 or at a position corresponding thereto (preferably or generally, valine);
(B-1_B-18) substitutions of isoleucine for an amino acid at a position 257 of the amino acid sequence set forth in SEQ ID NO: 2 or at a position corresponding thereto (preferably or generally, leucine), and phenylalanine for an amino acid at a position 110 of the amino acid sequence set forth in SEQ ID NO: 2 or at a position corresponding thereto (preferably or generally, valine); and
(B-1_B-19) substitutions of isoleucine for an amino acid at a position 257 of the amino acid sequence set forth in SEQ ID NO: 2 or at a position corresponding thereto (preferably or generally, leucine), and isoleucine for an amino acid at a position 118 of the amino acid sequence set forth in SEQ ID NO: 2 or at a position corresponding thereto (preferably or generally, cysteine).

A protein which contains the amino acid sequence of the protein (A) or (B) as a part of the amino acid sequence thereof and exhibits TE activity is preferably used for a TE that is used in the present invention. Further, an amino acid at N-terminal end of the protein is preferably methionine or leucine encoded by a start codon.

In the amino acid sequence constituting the above-described protein, a sequence other than the amino acid sequence of the above-described protein (A) or (B) can be appropriately selected within the range in which advantageous effects of the invention are not adversely affected. The examples thereof include the arbitrary amino acid sequence of 1^{st} to 57^{th} amino acids of the amino acid sequence set forth in SEQ ID NO: 17, an amino acid sequence in which 1 or several (preferably 1 or more and 20 or less, more preferably 1 or more and 15 or less, further preferably 1 or more and 10 or less, furthermore preferably 1 or more and 5 or less, and furthermore preferably 1 or more and 3 or less) mutations are introduced into the amino acid sequence, and the like. The examples of the mutation include deletion, substitution, insertion and addition of amino acids. These sequences are preferably added to the N-terminal side of the amino acid sequence of the protein (A) or (B).

Alternatively, a TE that is used in the present invention may be a protein consisting of the amino acid sequence in which a portion on the N-terminal side is deleted in an arbitrary position of the 2^{nd} to 57^{th} amino acids of the amino acid sequence set forth in SEQ ID NO: 17 in the amino acid sequence set forth in SEQ ID NO: 17. Moreover, a TE that is used in the present invention is also preferably a protein consisting of an amino acid sequence formed such that a signal peptide involved in transport or secretion of the protein is added to the amino acid sequence of the protein (A) or (B).

The TE activity of the protein can be confirmed by, for example, introducing a DNA produced by linking a gene encoding the protein to the downstream of a promoter which functions in a host cell such as Escherichia coli, into a host cell which lacks a fatty acid degradation system, culturing the thus-obtained cell under the conditions suitable for the expression of the introduced gene, and analyzing any change caused thereby in the fatty acid composition of the host cell or the cultured liquid by using a gas chromatographic analysis or the like. In this case, improving specificity to the medium-chain acyl-ACP in the TE variant can be confirmed by comparing a proportion of medium-chain fatty acids in the total amount of fatty acids with a proportion of a system in which the wild type TE is expressed.

Alternatively, the TE activity can be measured by introducing a DNA produced by linking a gene encoding the protein to the downstream of a promoter which functions in a host cell such as Escherichia coli, into a host cell, culturing the thus-obtained cell under the conditions suitable for the expression of the introduced gene, and subjecting a disruption liquid of the cell to a reaction which uses acyl-ACPs, as substrates, prepared according to the method of Yuan et al. (Proc. Natl. Acad. Sci. USA., 1995, vol. 92(23), p. 10639-10643).

A method of introducing the mutation into an amino acid sequence includes a method of, for example, introducing a mutation into a nucleotide sequence encoding the amino acid sequence. A method of introducing the mutation includes a method of introducing a site-specific mutation. Specific examples of the method of introducing the site-specific mutation include a method of utilizing the SOE-PCR, the ODA method, and the Kunkel method. Further, commercially available kits such as Site-Directed Mutagenesis System Mutan-Super Express Km kit (Takara Bio), Transformer TM Site-Directed Mutagenesis kit (Clontech Laboratories), and KOD-Plus-Mutagenesis Kit (TOYOBO) can also be utilized. Furthermore, a gene containing a desired mutation can also be obtained by introducing a genetic mutation at random, and then performing an evaluation of the enzyme activities and a gene analysis thereof by an appropriate method.

The proteins (A) and (B) can be obtained by chemical techniques, genetic engineering techniques or the like that are ordinarily carried out. For example, a natural product-derived protein can be obtained through isolation, purification and the like from Cuphea palustris. In addition, the proteins (A) and (B) can be obtained by artificial chemical synthesis based on the amino acid sequence set forth in SEQ ID NO: 2. Alternatively, as recombinant proteins, proteins (A) and (B) may also be produced by gene recombination technologies. In the case of producing a recombinant protein, the TE gene described below can be used.

Note that the plant such as Cuphea palustris can be obtained from culture collection such as private or public research institutes or the like.

Examples of genes encoding at least one protein selected form the group consisting of the proteins (A) and (B) (hereinafter, also referred to as “TE gene”) include a gene consisting of any one of the following DNAs (a) and (b). The DNA consisting of the nucleotide sequence set forth in SEQ ID NO: 1 encodes the protein consisting of the amino acid sequence set forth in SEQ ID NO: 2 (a protein consisting of a part of the amino acid sequence of a wild-type TE derived from Cuphea palustris). Further, the nucleotide sequence encoding the signal sequence (amino acid sequence of the 1^{st} to 57^{th} amino acids of the amino acid sequence set forth in SEQ ID NO: 17) corresponds to the nucleotide sequence of the 1^{st} to 171^{st} nucleotides of the nucleotide sequence set forth in SEQ ID NO: 16. Hereinafter, a gene consisting of the DNA (a) is also referred to as “CpTE gene”.

(a) a DNA consisting of the nucleotide sequence set forth in SEQ ID NO: 1;
(b) a DNA consisting of a nucleotide sequence having 60% or more identity with the nucleotide sequence set forth in SEQ ID NO: 1, and encoding the protein having TE activity.

In the DNA (b), the identity with the nucleotide sequence set forth in SEQ ID NO: 1 is 60% or more, preferably 65% or more, more preferably 70% or more, further preferably 75% or more, further preferably 80% or more, further preferably 85% or more, further preferably 90% or more, further preferably 93% or more, further preferably 95% or more, further preferably 96% or more, further preferably 97% or more, further preferably 98% or more, and furthermore preferably 99% or more, in view of TE activity.

Further, the DNA (b) is also preferably a DNA in which 1 or several (for example 1 or more and 427 or less, preferably 1 or more and 373 or less, more preferably 1 or more and 320 or less, further preferably 1 or more and 267 or less, further preferably 1 or more and 213 or less, further preferably 1 or more and 160 or less, further preferably 1 or more and 106 or less, further preferably 1 or more and 74 or less, further preferably 1 or more and 53 or less, further preferably 1 or more and 42 or less, further preferably 1 or more and 32 or less, further preferably 1 or more and 21 or less, and furthermore preferably 1 or more and 10 or less) nucleotides are deleted, substituted, inserted or added to the nucleotide sequence set forth in SEQ ID NO: 1, and encoding the protein (A) or (B) having TE activity.

Furthermore, the DNA (b) is also preferably a DNA capable of hybridizing with a DNA consisting of the nucleotide sequence complementary with the DNA (a) under a stringent condition, and encoding the protein (A) or (B) having TE activity.

Specific examples of the DNA (b) that is preferably used in the present invention include a DNA in which nucleotides at specific positions in the DNA coding the amino acid sequence of the protein (B) are substituted, and a DNA containing the nucleotide substitutions.

The DNA (b) is also preferably a DNA consisting of a nucleotide sequence having at least one nucleotide substitution selected from the group consisting of the following (b-1) to (b-11). The following (b-1) to (b-11) are nucleotide substations corresponding to the amino acid substitutions of the (B-1) to (B-11). Specifically, the nucleotide substitutions of (b-1), (b-2), (b-3), (b-4), (b-5), (b-6), (b-7), (b-8), (b-9), (b-10), and (b-11) respectively correspond to the amino acid substitutions of (B-1), (B-2), (B-3), (B-4), (B-5), (B-6), (B-7), (B-8), (B-9), (B-10), and (B-11).

(b-1) substitution of nucleotides encoding isoleucine for nucleotides at positions 769 to 771 of the nucleotide sequence set forth in SEQ ID NO: 1, or at positions corresponding thereto;
(b-2) substitution of nucleotides encoding arginine for nucleotides at positions 751 to 753 of the nucleotide sequence set forth in SEQ ID NO: 1, or at positions corresponding thereto;
(b-3) substitution of nucleotides encoding lysine for nucleotides at positions 751 to 753 of the nucleotide sequence set forth in SEQ ID NO: 1, or at positions corresponding thereto;
(b-4) substitution of nucleotides encoding histidine for nucleotides at positions 751 to 753 of the nucleotide sequence set forth in SEQ ID NO: 1, or at positions corresponding thereto;
(b-5) substitution of nucleotides encoding isoleucine for nucleotides at positions 760 to 762 of the nucleotide sequence set forth in SEQ ID NO: 1, or at positions corresponding thereto;
(b-6) substitution of nucleotides encoding tyrosine for nucleotides at positions 760 to 762 of the nucleotide sequence set forth in SEQ ID NO: 1, or at positions corresponding thereto;
(b-7) substitution of nucleotides encoding methionine for nucleotides at positions 769 to 771 of the nucleotide sequence set forth in SEQ ID NO: 1, or at positions corresponding thereto;
(b-8) substitution of nucleotides encoding valine for nucleotides at positions 769 to 771 of the nucleotide sequence set forth in SEQ ID NO: 1, or at positions corresponding thereto;
(b-9) substitution of nucleotides encoding phenylalanine for nucleotides at positions 769 to 771 of the nucleotide sequence set forth in SEQ ID NO: 1, or at positions corresponding thereto;
(b-10) substitution of nucleotides encoding cysteine for nucleotides at positions 796 to 798 of the nucleotide sequence set forth in SEQ ID NO: 1, or at positions corresponding thereto; and
(b-11) substitution of nucleotides encoding tyrosine for nucleotides at positions 811 to 813 of the nucleotide sequence set forth in SEQ ID NO: 1, or at positions corresponding thereto.

The DNA (b) preferably has at least one nucleotide substitution selected from the group consisting of the following (b-12) to (b-19), in addition to at least one nucleotide substitution selected from the group consisting of the (b-1) to (b-11). The following (b-12) to (b-19) are nucleotide substitutions corresponding to the amino acid substitutions of the (B-12) to (B-19). Specifically, the nucleotide substitutions of (b-12), (b-13), (b-14), (b-15), (b-16), (b-17), (b-18) and (b-19) respectively correspond to the amino acid substitutions of (B-12), (B-13), (B-14), (B-15), (B-16), (B-17), (B-18) and (B-19).

(b-12) substitution of nucleotides encoding isoleucine for nucleotides at positions 316 to 318 of the nucleotide sequence set forth in SEQ ID NO: 1, or at positions corresponding thereto;
(b-13) substitution of nucleotides encoding lysine for nucleotides at positions 322 to 324 of the nucleotide sequence set forth in SEQ ID NO: 1, or at positions corresponding thereto;
(b-14) substitution of nucleotides encoding arginine for nucleotides at positions 322 to 324 of the nucleotide sequence set forth in SEQ ID NO: 1, or at positions corresponding thereto;
(b-15) substitution of nucleotides encoding isoleucine for nucleotides at positions 328 to 330 of the nucleotide sequence set forth in SEQ ID NO: 1, or at positions corresponding thereto;
(b-16) substitution of nucleotides encoding methionine for nucleotides at positions 328 to 330 of the nucleotide sequence set forth in SEQ ID NO: 1, or at positions corresponding thereto;
(b-17) substitution of nucleotides encoding leucine for nucleotides at positions 328 to 330 of the nucleotide sequence set forth in SEQ ID NO: 1, or at positions corresponding thereto;
(b-18) substitution of nucleotides encoding phenylalanine for nucleotides at positions 328 to 330 of the nucleotide sequence set forth in SEQ ID NO: 1, or at positions corresponding thereto; and
(b-19) substitution of nucleotides encoding isoleucine for nucleotides at positions 352 to 354 of the nucleotide sequence set forth in SEQ ID NO: 1, or at positions corresponding thereto.

In the present invention, the DNA (b) more preferably has the nucleotide substitution of (b-1) and at least one nucleotide substitution selected from the group consisting of the (b-12) to (b-19). Namely, in the present invention, the DNA (b) preferably has the nucleotide substitution selected from the group consisting of the following (b-1_b-12) to (b-1_b-19):

(b-1_b-12) substitutions of nucleotides encoding isoleucine for nucleotides at positions 769 to 771 of the nucleotide sequence set forth in SEQ ID NO: 1 or at positions corresponding thereto, and nucleotides encoding isoleucine for nucleotides at positions 316 to 318 of the nucleotide sequence set forth in SEQ ID NO: 1 or at positions corresponding thereto;
(b-1_b-13) substitutions of nucleotides encoding isoleucine for nucleotides at positions 769 to 771 of the nucleotide sequence set forth in SEQ ID NO: 1 or at positions corresponding thereto, and nucleotides encoding lysine for nucleotides at positions 322 to 324 of the nucleotide sequence set forth in SEQ ID NO: 1 or at positions corresponding thereto;
(b-1_b-14) substitutions of nucleotides encoding isoleucine for nucleotides at positions 769 to 771 of the nucleotide sequence set forth in SEQ ID NO: 1 or at positions corresponding thereto, and nucleotides encoding arginine for nucleotides at positions 322 to 324 of the nucleotide sequence set forth in SEQ ID NO: 1 or at positions corresponding thereto;
(b-1_b-15) substitutions of nucleotides encoding isoleucine for nucleotides at positions 769 to 771 of the nucleotide sequence set forth in SEQ ID NO: 1 or at positions corresponding thereto, and nucleotides encoding isoleucine for nucleotides at positions 328 to 330 of the nucleotide sequence set forth in SEQ ID NO: 1 or at positions corresponding thereto;
(b-1_b-16) substitutions of nucleotides encoding isoleucine for nucleotides at positions 769 to 771 of the nucleotide sequence set forth in SEQ ID NO: 1 or at positions corresponding thereto, and nucleotides encoding methionine for nucleotides at positions 328 to 330 of the nucleotide sequence set forth in SEQ ID NO: 1 or nucleotides at positions corresponding thereto;
(b-1_b-17) substitutions of nucleotides encoding isoleucine for nucleotides at positions 769 to 771 of the nucleotide sequence set forth in SEQ ID NO: 1 or at positions corresponding thereto, and nucleotides encoding leucine for nucleotides at positions 328 to 330 of the nucleotide sequence set forth in SEQ ID NO: 1 or at positions corresponding thereto;
(b-1_b-18) substitutions of nucleotides encoding isoleucine for nucleotides at positions 769 to 771 of the nucleotide sequence set forth in SEQ ID NO: 1 or at positions corresponding thereto, and nucleotides encoding phenylalanine for nucleotides at positions 328 to 330 of the nucleotide sequence set forth in SEQ ID NO: 1 or at positions corresponding thereto; and
(b-1_b-19) substitutions of nucleotides encoding isoleucine for nucleotides at positions 769 to 771 of the nucleotide sequence set forth in SEQ ID NO: 1 or at positions corresponding thereto, and nucleotides encoding isoleucine for nucleotides at positions 352 to 354 of the nucleotide sequence set forth in SEQ ID NO: 1 or at positions corresponding thereto.

A method of introducing the mutation such as deletion, substitution, addition, and insertion into a nucleotide sequence includes, for example, a method of introducing a site-specific mutation. Specific examples of the method of introducing the site-specific mutation include a method of utilizing the Splicing overlap extension (SOE)-PCR (Gene, 1989, vol. 77, p. 61-68), the ODA method (Gene, 1995, 152, 271-276), and the Kunkel method (Proc. Natl. Acad. Sci. USA, 1985, vol. 82, p. 488). Further, commercially available kits such as Site-Directed Mutagenesis System Mutan-Super Express Km kit (Takara Bio), Transformer TM Site-Directed Mutagenesis kit (Clontech Laboratories), and KOD-Plus-Mutagenesis Kit (TOYOBO) can also be utilized. Furthermore, a gene containing a desired mutation can also be obtained by introducing a genetic mutation at random, and then performing an evaluation of the enzyme activities and a gene analysis thereof by an appropriate method.

A gene encoding the CpTE variant (hereinafter, also referred to as “CpTE variant gene”) can be obtained by genetic engineering techniques that are ordinarily carried out. For example, the CpTE variant gene can be artificially synthesized based on the amino acid sequence set forth in SEQ ID NO: 2 or the nucleotide sequence set forth in SEQ ID NO: 1. Further, the CpTE variant gene can also be obtained by cloning from Nannochloropsis oculata. The cloning can be carried out by, for example, the methods described in Molecular Cloning: A LABORATORY MANUAL THIRD EDITION [Joseph Sambrook, David W. Russell, Cold Spring Harbor Laboratory Press (2001)].

FabG is a protein (enzyme) which has β-ketoacyl-ACP reductase activity (hereinafter, also referred to as “FabG activity”). FabG catalyzes, depending on NADPH or NADH as a coenzyme, a reaction in which an acetoacetyl ACP is reduced to produce a β-hydroxyacyl-ACP. In the present specification, FabG activity means an activity of reducing an acetoacetyl-ACP.

In the present invention, the protein (C) or (D) is used for a FabG.

The protein (C) consisting of the amino acid sequence set forth in SEQ ID NO: 4 is a FabG derived from Cupriavidus taiwanensis.

Hereinafter, the protein (C) is also referred to as “CtFabG”.

From the viewpoint of competition of energy, the FabG used in the invention is preferably a NADH-type FabG. Thus, FabG activity of the FabG in the present invention is preferably an activity of catalyzing a reaction in which an acetoacetyl ACP is reduced, in a NADH-dependent manner, to produce a β-hydroxyacyl-ACP (hereinafter, also referred to as “NADH-type FabG activity”).

The protein (C) used in the present invention has NADH-type FabG activity.

Whether the FabG used in the present invention utilizes NADH as a coenzyme can be confirmed by evaluating whether the FabG exhibits, for example, reductase activity for acetoacetyl-CoA in the presence of NADH or NADPH.

The protein (D) is a protein consisting of an amino acid sequence having 60% or more identity with the amino acid sequence set forth in SEQ ID NO: 4, and having FabG activity.

In the protein (D), the identity with the amino acid sequence set forth in SEQ ID NO: 4 is 60% or more, preferably 65% or more, more preferably 70% or more, further preferably 75% or more, further preferably 80% or more, further preferably 85% or more, further preferably 90% or more, further preferably 93% or more, further preferably 95% or more, further preferably 96% or more, further preferably 97% or more, further preferably 98% or more, and furthermore preferably 99% or more, in view of FabG activity.

Further, specific examples of the protein (D) include a protein in which 1 or several (for example 1 or more and 98 or less, preferably 1 or more and 86 or less, more preferably 1 or more and 73 or less, further preferably 1 or more and 61 or less, furthermore preferably 1 or more and 49 or less, furthermore preferably 1 or more and 36 or less, furthermore preferably 1 or more and 24 or less, furthermore preferably 1 or more and 17 or less, furthermore preferably 1 or more and 12 or less, furthermore preferably 1 or more and 9 or less, furthermore preferably 1 or more and 7 or less, furthermore preferably 1 or more and 4 or less, and furthermore preferably 1 or 2) amino acids are deleted, substituted, inserted or added to the amino acid sequence set forth in SEQ ID NO: 4, and having FabG activity.

Moreover, the protein (D) also preferably includes a protein consisting of an amino acid sequence formed such that a signal peptide engaging in transport or secretion of the protein is added to the amino acid sequence of the protein (C) or (D).

The FabG activity of the protein can be confirmed by, for example, introducing a DNA produced by linking a gene encoding the protein to the downstream of a promoter which functions in a host cell such as Escherichia coli, into a host cell which lacks a fatty acid degradation system, culturing the thus-obtained cell under the conditions suitable for the expression of the introduced gene, and analyzing any change caused thereby in the fatty acid composition of the host cell or the cultured liquid by using a gas chromatographic analysis or the like.

Alternatively, the FabG activity can be measured by introducing a DNA produced by linking a gene encoding the protein to the downstream of a promoter which functions in a host cell such as Escherichia coli, into a host cell, culturing the thus-obtained cell under the conditions suitable for the expression of the introduced gene, and subjecting a disruption liquid of the cell to a reaction which uses acyl-ACPs, as substrates, prepared according to the method of Yuan et al. (Proc. Natl. Acad. Sci. USA., 1995, vol. 92(23), p. 10639-10643).

The proteins (C) and (D) can be obtained by chemical techniques, genetic engineering techniques or the like that are ordinarily carried out. For example, a natural product-derived protein can be obtained through isolation, purification and the like from Cupriavidus taiwanensis. In addition, the proteins (C) and (D) can be obtained by artificial chemical synthesis based on the amino acid sequence set forth in SEQ ID NO: 4. Alternatively, as recombinant proteins, proteins (C) and (D) may also be produced by gene recombination technologies. In the case of producing a recombinant protein, the FabG gene described below can be used.

Note that the bacteria such as Cupriavidus taiwanensis can be obtained from culture collection such as private or public research institutes or the like.

Specific examples of genes encoding at least one protein selected form the group consisting of the proteins (C) and (D) (hereinafter, also referred to as “FabG gene”) include a gene consisting of any one of the following DNAs (c) and (d). The DNA consisting of the nucleotide sequence set forth in SEQ ID NO: 3 encodes the protein consisting of the amino acid sequence set forth in SEQ ID NO: 4 (CtFabG). Hereinafter, a gene consisting of the DNA (c) is also referred to as “CtFabG gene”.

(c) a DNA consisting of the nucleotide sequence set forth in SEQ ID NO: 3; and
(d) a DNA consisting of a nucleotide sequence having 60% or more identity with the nucleotide sequence set forth in SEQ ID NO: 3, and encoding the protein having FabG activity.

In the DNA (d), the identity with the nucleotide sequence set forth in SEQ ID NO: 3 is 60% or more, preferably 65% or more, more preferably 70% or more, further preferably 75% or more, further preferably 80% or more, further preferably 85% or more, further preferably 90% or more, further preferably 93% or more, further preferably 95% or more, further preferably 96% or more, further preferably 97% or more, further preferably 98% or more, and furthermore preferably 99% or more, in view of FabG activity.

Further, the DNA (d) is also preferably a DNA in which 1 or several (for example 1 or more and 296 or less, preferably 1 or more and 259 or less, more preferably 1 or more and 222 or less, further preferably 1 or more and 185 or less, further preferably 1 or more and 148 or less, further preferably 1 or more and 111 or less, further preferably 1 or more and 74 or less, further preferably 1 or more and 51 or less, further preferably 1 or more and 37 or less, further preferably 1 or more and 29 or less, further preferably 1 or more and 22 or less, further preferably 1 or more and 14 or less, and furthermore preferably 1 or more and 7 or less) nucleotides are deleted, substituted, inserted or added to the nucleotide sequence set forth in SEQ ID NO: 3, and encoding a protein (C) or (D) having FabG activity. Furthermore, the DNA (d) is also preferably a DNA capable of hybridizing with a DNA consisting of the nucleotide sequence complementary with the DNA (c) under a stringent condition, and encoding the protein (C) or (D) having FabG activity.

Moreover, a DNA encoding the FabG used in the present invention also may be a gene consisting of a nucleotide sequence wherein a DNA encoding a signal peptide involved in transport or secretion of a protein, or an amino acid sequence or the like which is well known to increase stability of a protein is added to the nucleotide sequence of the DNA (c) or (d).

A gene encoding FabG can be obtained by genetic engineering techniques that are ordinarily carried out. For example, FabG gene can be artificially synthesized based on the amino acid sequence set forth in SEQ ID NO: 4 or the nucleotide sequence set forth in SEQ ID NO: 3. Further, FabG gene can also be obtained by cloning from Cupriavidus taiwanensis. The cloning can be carried out by, for example, the methods described in Molecular Cloning: A LABORATORY MANUAL THIRD EDITION [Joseph Sambrook, David W. Russell, Cold Spring Harbor Laboratory Press (2001)].

In the transformant of the present invention, a gene encoding the protein (A) or (B), and a gene encoding the protein (C) or (D) are introduced into a host cell, and expression thereof is enhanced.

As described above, it is known that productivity of medium-chain fatty acids having 8 or 10 carbon atoms in a transformant obtained is improved by introducing a gene encoding a TE derived from plants belonging to the genus Cuphea or a variant thereof into a host. In contrast to this finding, productivity of medium-chain fatty acids having 8 or 10 carbon atoms and whole fatty acids is significantly improved in a transformant prepared by introducing a TE gene and a FabG gene into a host, as compared with that in a transformant prepared by introducing only a TE gene into a host.

Therefore, productivity of medium-chain fatty acids or lipids containing the same as components produced in a cell of the transformant is improved by culturing the transformant of the present invention.

The transformant of the present invention can be prepared by introducing a gene encoding the protein (A) or (B), and a gene encoding the protein (C) or (D) into a host according to an ordinarily method. Specifically, the transformant can be produced by preparing a recombinant vector or a gene expression cassette which is capable of expressing the TE gene and the FabG gene in a host cell, introducing this vector or cassette into a host cell, and thereby transforming the host cell.

The host for the transformant can be appropriately selected from ordinarily used hosts. A host that can be used in the present invention is, from a viewpoint of supplying a coenzyme which is used by a FabG, preferably a microorganism, and more preferably a microorganism having only a NADPH-type FabG as a FabG. Specific examples of microorganism having a NADPH-type FabG as a FabG include Escherichia coli, Pseudomonas aeruginosa, and Bacillus subtilis. Among them, from a viewpoint of lipid productivity, Escherichia coli and Bacillus subtilis are preferable, and Escherichia coli is more preferable.

A vector for use as the plasmid for gene expression or a vector containing the gene expression cassette (plasmid) may be any vector capable of introducing the gene encoding the target protein into a host, and expressing the target gene in the host cell. For example, a vector which has expression regulation regions such as a promoter and a terminator in accordance with the type of the host to be used, and has a replication initiation point, a selection marker or the like, can be used. Furthermore, the vector may also be a vector such as a plasmid capable of self-proliferation and self-replication outside the chromosome, or may also be a vector which is incorporated into the chromosome.

Specific examples of the vector that can be used preferably in the present invention include, in the case of using a microorganism as the host, pBluescript (pBS) II SK(−) (manufactured by Stratagene), a pSTV-based vector (manufactured by Takara Bio), a pUC-based vector (manufactured by Takara Shuzo), a pET-based vector (manufactured by Takara Bio), a pGEX-based vector (manufactured by GE Healthcare), a pCold-based vector (manufactured by Takara Bio), pHY300PLK (manufactured by Takara Bio), pUB110 (1986, Plasmid 15(2), p. 93-103), pBR322 (manufactured by Takara Bio), pRS403 (manufactured by Stratagene), pMW218/219 (manufactured by Nippon Gene), a pRI-based vector (manufactured by Takara Bio), a pBI-based vector (manufactured by Clontech), and an IN3-based vector (manufactured by Inplanta Innovations Inc.). In particular, in the case of using Escherichia coli as the host, pBluescript II SK(−) or pMW218/219 is preferably used.

Introduction of the gene encoding a target protein to the vector can be conducted by an ordinary technique such as restriction enzyme treatment and ligation.

A kind of promoter regulating the expression of the gene encoding a target protein, which is introduced into the expression vector, can also be appropriately selected according to a kind of the host to be used. Specific examples of the promoter that can be preferably used in the present invention include lac promoter, trp promoter, tac promoter, trc promoter, T7 promoter, SpoVG promoter, a promoter that relates to a substance that can be induced by addition of isopropyl β-D-1-thiogalactopyranoside (IPTG), Rubisco operon (rbc), PSI reaction center protein (psaAB), D1 protein of PSII (psbA), cauliflower mosaic virus 35S RNA promoter, promoters for housekeeping genes (e.g., tubulin promoter, actin promoter and ubiquitin promoter), Brassica napus or Brassica rapa-derived Napin gene promoter, plant-derived Rubisco promoter, a promoter of a violaxanthin/(chlorophyll a)-binding protein gene derived from the genus Nannochloropsis (VCP1 promoter, VCP2 promoter) (Proceedings of the National Academy of Sciences of the United States of America, 2011, vol. 108(52)), a promoter of an oleosin-like protein LDSP (lipid droplet surface protein) gene derived from the genus Nannochloropsis (PLOS Genetics, 2012, vol. 8(11): e1003064. DOI: 10.1371), and a promoter of an rrnA operon gene encoding a ribosomal RNA.

Moreover, a kind of selection marker for confirming introduction of the gene encoding a target protein can also be appropriately selected according to a kind of the host to be used. Examples of the selection marker that can be preferably used in the present invention include drug resistance genes such as an ampicillin resistance gene, a chloramphenicol resistance gene, an erythromycin resistance gene, a neomycin resistance gene, a kanamycin resistance gene, a spectinomycin resistance gene, a tetracycline resistance gene, a blasticidin S resistance gene, a bialaphos resistance gene, a zeocin resistance gene, a paromomycin resistance gene, and a hygromycin resistance gene. Further, it is also possible to use a deletion of an auxotrophy-related gene or the like as the selection marker gene.

The method for transformation can be appropriately selected from ordinary techniques according to a kind of the host to be used. Examples of the method for transformation include a transformation method of using calcium ion, a general competent cell transformation method, a protoplast transformation method, an electroporation method, an LP transformation method, a method of using Agrobacterium, a particle gun method, and the like.

The selection of a transformant having a target gene fragment introduced therein can be carried out by utilizing the selection marker or the like. For example, the selection can be carried out by using an indicator whether a transformant acquires the drug resistance as a result of introducing a drug resistance gene into a host cell together with a target DNA fragment upon the transformation. Further, the introduction of a target DNA fragment can also be confirmed by PCR method using a genome as a template or the like.

The TE gene and the FabG gene to be introduced into each of hosts are preferably optimized in codon in accordance with use frequency of codon in the host to be used. Information of codons used in each of organisms is available from Codon Usage Database (www.kazusa.or.jp/codon/).

In the transformant of the present invention, productivity of medium-chain fatty acids or lipids containing the same as components is significantly improved, in comparison with that in a transformant into which only TE gene is introduced. Therefore, the transformant of the present invention can be preferably applied to production of fatty acids having specific number of carbon atoms or lipids, particularly medium-chain fatty acids or lipids containing the same as components, preferably fatty acids having 8 or more and 10 or less carbon atoms or lipids containing the same as components, more preferably fatty acids having 8 or 10 carbon atoms or lipids containing the same as components, further preferably saturated fatty acids having 8 or 10 carbon atoms (caprylic acid or capric acid) or lipids containing the same as components, furthermore preferably saturated fatty acids having 8 carbon atoms (caprylic acid) or lipids containing the same as components.

Hereinafter, in the present specification, a cell into which a gene encoding at least one protein selected from the group consisting of the proteins (A) to (D) is introduced is also referred to as the “transformant”. On the other hand, a cell into which none of a gene encoding the proteins (A) to (D) is introduced is also referred to as the “host” or “wild type strain”.

In the transformant of the present invention, productivity of medium-chain fatty acids or lipids containing the same as components is improved in comparison with that in the host in which expression of the protein (A) or (B), and expression of the protein (C) or (D) is not enhanced. Accordingly, when the transformant of the present invention is cultured under suitable conditions and then medium-chain fatty acids or lipids containing the same as components are collected from an obtained cultured product, the medium-chain fatty acids or the lipids containing the same as components can be efficiently produced. Herein, the term “cultured product” means liquid medium and a transformant subjected to cultivation.

The culture condition of the transformant of the present invention can be appropriately selected in accordance with the type of the host, and any ordinary used culture condition for the host can be employed. For example, glycerol is preferably used as a carbon source.

Culturing of Escherichia coli may be carried out, for example, in LB medium or Overnight Express Instant TB Medium (Novagen) at 30 to 37° C. for half a day to 1 day.

A method of collecting the lipids from the cultured product is appropriately selected from an ordinary method. For example, lipid components can be isolated and collected from the above-described cultured product by means of filtration, centrifugation, cell disruption, gel filtration chromatography, ion exchange chromatography, chloroform/methanol extraction, hexane extraction, ethanol extraction, or the like. In the case of carrying out the larger scale culturing, lipids can be obtained by collecting oil components from the cultured product through pressing or extraction, and then performing general purification processes such as degumming, deacidification, decoloration, dewaxing, and deodorization. After lipid components are isolated as such, the isolated lipids are hydrolyzed, and thereby fatty acids can be obtained. Specific examples of the method of isolating fatty acids from lipid components include a method of treating the lipid components at a high temperature of about 70° C. in an alkaline solution, a method of performing a lipase treatment, and a method of degrading the lipid components using high-pressure hot water.

Moreover, in the case of using a transformant prepared by using, as a host, Escherichia coli prepared by causing loss of the function of a β-oxidation pathway being a fatty acid degradation pathway, produced lipids are secreted to the outside of cells. Therefore, it is unnecessary to destroy bacterial cells in order to collect the lipid, and the cells remaining after collecting the lipid can be repeatedly used for production of the lipid.

The lipids produced in the production method of the present invention preferably contain fatty acids or fatty acid compounds, and more preferably contain fatty acids or fatty acid ester compounds, in view of usability thereof. The fatty acid ester compound is preferably at least one kind selected from the group consisting of MAG, DAG, and TAG, and more preferably TAG.

In view of usability for a surfactant or the like, and from a nutritional viewpoint, the fatty acid or the ester compound thereof contained in the lipid is preferably a medium-chain fatty acid or an ester compound thereof. Specifically, the fatty acid or the ester compound thereof contained in the lipid is preferably a fatty acid having 8 or more and 10 or less carbon atoms or an ester compound thereof, more preferably a fatty acid having 8 or 10 carbon atoms or an ester compound thereof, more preferably a saturated fatty acid having 8 or 10 carbon atoms (caprylic acid or capric acid) or an ester compound thereof, more preferably a saturated fatty acid having 8 carbon atoms (caprylic acid) or an ester compound thereof.

From a viewpoint of productivity, the fatty acid ester compound is preferably a simple lipid or a complex lipid, more preferably a simple lipid, and further preferably a triacylglycerol.

The lipid obtained by the production method of the present invention can be utilized for food, as well as a plasticizer, an emulsifier incorporated into cosmetic products or the like, a cleansing agent such as a soap or a detergent, a fiber treatment agent, a hair conditioning agent, a disinfectant or an antiseptic.

With regard to the embodiments described above, the present invention also discloses methods of producing lipids, methods of enhancing lipid productivity, transformants and methods of preparing the same, described below.

<1> A method of producing lipids, containing the steps of:

culturing a transformant in which the expression of a gene encoding the following protein (A) or (B), and the expression of a gene encoding the following protein (C) or (D) is enhanced, and

producing medium-chain fatty acids or the lipids containing the same as components:

(A) a protein consisting of the amino acid sequence set forth in SEQ ID NO: 2;
(B) a protein consisting of an amino acid sequence having 60% or more, preferably 70% or more, more preferably 75% or more, further preferably 80% or more, furthermore preferably 85% or more, furthermore preferably 90% or more, furthermore preferably 93% or more, furthermore preferably 95% or more, furthermore preferably 96% or more, furthermore preferably 97% or more, furthermore preferably 98% or more, and furthermore preferably 99% or more identity with the amino acid sequence set forth in SEQ ID NO: 2, and having TE activity;
(C) a protein consisting of the amino acid sequence set forth in SEQ ID NO: 4; and
(D) a protein consisting of an amino acid sequence having 60% or more, preferably 70% or more, more preferably 75% or more, further preferably 80% or more, furthermore preferably 85% or more, furthermore preferably 90% or more, furthermore preferably 93% or more, furthermore preferably 95% or more, furthermore preferably 96% or more, furthermore preferably 97% or more, furthermore preferably 98% or more, and furthermore preferably 99% or more identity with the amino acid sequence set forth in SEQ ID NO: 4, and having FabG activity.
<2> A method of improving lipid productivity, containing the steps of:

enhancing the expression of a gene encoding the protein (A) or (B), and the expression of a gene encoding the protein (C) or (D), and

improving the productivity of medium-chain fatty acids or the lipids containing the same as components, produced in a cell of a transformant.

<3> A method of improving lipid productivity, containing the steps of:

enhancing the expression of a gene encoding the protein (A) or (B), and a gene encoding the protein (C) or (D), and

improving total amount of fatty acids produced in a cell of a transformant.

<4> The method described in any one of the above items <1> to <3>, wherein a gene encoding the protein (A) or (B) and a gene encoding the protein (C) or (D) are introduced into a host cell to enhance expression of the genes.
<5> A method of producing lipids, containing the steps of:

culturing a transformant wherein a gene encoding the protein (A) or (B), and a gene encoding the protein (C) or (D) are introduced into a host cell, and

producing medium-chain fatty acids or the lipids containing the same as components.

<6> A method of improving lipid productivity, containing the steps of:

culturing a transformant wherein a gene encoding the protein (A) or (B), and a gene encoding the protein (C) or (D) are introduced into a host cell, and

improving the productivity of medium-chain fatty acids or the lipids containing the same as components, produced in a cell of the transformant.

<7> A method of improving lipid productivity, containing the steps of:

culturing a transformant wherein a gene encoding the protein (A) or (B), and a gene encoding the protein (C) or (D) are introduced into a host cell, and

improving total amount of fatty acids produced in a cell of the transformant.

<8> The method described in any one of the above items <1> to <7>, wherein the protein (B) is a protein consisting of an amino acid sequence in which 1 or several amino acids, preferably 1 or more and 142 or less amino acids, more preferably 1 or more and 124 or less amino acids, further preferably 1 or more and 106 or less amino acids, furthermore preferably 1 or more and 88 or less amino acids, furthermore preferably 1 or more and 71 or less amino acids, furthermore preferably 1 or more and 53 or less amino acids, furthermore preferably 1 or more and 35 or less amino acids, furthermore preferably 1 or more and 24 or less amino acids, furthermore preferably 1 or more and 17 or less amino acids, furthermore preferably 1 or more and 14 or less amino acids, furthermore preferably 1 or more and 10 or less amino acids, furthermore preferably 1 or more and 7 or less amino acids, and furthermore preferably 1 or more and 3 or less amino acids, are deleted, substituted, inserted or added to the amino acid sequence of the protein (A), and having TE activity.
<9> The method described in any one of the above items <1> to <8>, wherein the protein (B) is a protein consisting of an amino acid sequence having at least one amino acid substitution selected from the group consisting of the following (B-1) to (B-11), preferably the amino acid substitution of (B-1):
(B-1) substitution of isoleucine for an amino acid at a position 257 of the amino acid sequence set forth in SEQ ID NO: 2 or at a position corresponding thereto (preferably or generally, leucine);
(B-2) substitution of arginine for an amino acid at a position 251 of the amino acid sequence set forth in SEQ ID NO: 2 or at a position corresponding thereto (preferably or generally, threonine);
(B-3) substitution of lysine for an amino acid at a position 251 of the amino acid sequence set forth in SEQ ID NO: 2 or at a position corresponding thereto (preferably or generally, threonine);
(B-4) substitution of histidine for an amino acid at a position 251 of the amino acid sequence set forth in SEQ ID NO: 2 or at a position corresponding thereto (preferably or generally, threonine);
(B-5) substitution of isoleucine for an amino acid at a position 254 of the amino acid sequence set forth in SEQ ID NO: 2 or at a position corresponding thereto (preferably or generally, tryptophan);
(B-6) substitution of tyrosine for an amino acid at a position 254 of the amino acid sequence set forth in SEQ ID NO: 2 or at a position corresponding thereto (preferably or generally, tryptophan);
(B-7) substitution of methionine for an amino acid at a position 257 of the amino acid sequence set forth in SEQ ID NO: 2 or at a position corresponding thereto (preferably or generally, leucine);
(B-8) substitution of valine for an amino acid at a position 257 of the amino acid sequence set forth in SEQ ID NO: 2 or at a position corresponding thereto (preferably or generally, leucine);
(B-9) substitution of phenylalanine for an amino acid at a position 257 of the amino acid sequence set forth in SEQ ID NO: 2 or at a position corresponding thereto (preferably or generally, leucine);
(B-10) substitution of cysteine for an amino acid at a position 266 of the amino acid sequence set forth in SEQ ID NO: 2 or at a position corresponding thereto (preferably or generally, valine); and
(B-11) substitution of tyrosine for an amino acid at a position 271 of the amino acid sequence set forth in SEQ ID NO: 2 or at a position corresponding thereto (preferably or generally, tryptophan).
<10> The method described in the above item <9>, wherein the protein (B) is a protein consisting of an amino acid sequence having at least one amino acid substitution selected from the group consisting of the following (B-12) to (B-19), in addition to at least one amino acid substitution selected from the group consisting of the (B-1) to (B-11):
(B-12) substitution of isoleucine for an amino acid at a position 106 of the amino acid sequence set forth in SEQ ID NO: 2 or at a position corresponding thereto (preferably or generally, valine);
(B-13) substitution of lysine for an amino acid at a position 108 of the amino acid sequence set forth in SEQ ID NO: 2 or at a position corresponding thereto (preferably or generally, asparagine);
(B-14) substitution of arginine for an amino acid at a position 108 of the amino acid sequence set forth in SEQ ID NO: 2 or at a position corresponding thereto (preferably or generally, asparagine);
(B-15) substitution of isoleucine for an amino acid at a position 110 of the amino acid sequence set forth in SEQ ID NO: 2 or at a position corresponding thereto (preferably or generally, valine);
(B-16) substitution of methionine for an amino acid at a position 110 of the amino acid sequence set forth in SEQ ID NO: 2 or at a position corresponding thereto (preferably or generally, valine);
(B-17) substitution of leucine for an amino acid at a position 110 of the amino acid sequence set forth in SEQ ID NO: 2 or at a position corresponding thereto (preferably or generally, valine);
(B-18) substitution of phenylalanine for an amino acid at a position 110 of the amino acid sequence set forth in SEQ ID NO: 2 or at a position corresponding thereto (preferably or generally, valine); and
(B-19) substitution of isoleucine for an amino acid at a position 118 of the amino acid sequence set forth in SEQ ID NO: 2 or at a position corresponding thereto (preferably or generally, cysteine).
<11> The method described in any one of the above items <1> to <10>, wherein the protein (B) is a protein consisting of an amino acid sequence having an amino acid substitution selected from the group consisting of the following (B-1_B-12) to (B-1_B-19):
(B-1_B-12) substitutions of isoleucine for an amino acid at a position 257 of the amino acid sequence set forth in SEQ ID NO: 2 or at a position corresponding thereto (preferably or generally, leucine), and isoleucine for an amino acid at a position 106 of the amino acid sequence set forth in SEQ ID NO: 2 or at a position corresponding thereto (preferably or generally, valine);
(B-1_B-13) substitutions of isoleucine for an amino acid at a position 257 of the amino acid sequence set forth in SEQ ID NO: 2 or at a position corresponding thereto (preferably or generally, leucine), and lysine for an amino acid at a position 108 of the amino acid sequence set forth in SEQ ID NO: 2 or at a position corresponding thereto (preferably or generally, asparagine);
(B-1_B-14) substitutions of isoleucine for an amino acid at a position 257 of the amino acid sequence set forth in SEQ ID NO: 2 or at a position corresponding thereto (preferably or generally, leucine), and arginine for an amino acid at a position 108 of the amino acid sequence set forth in SEQ ID NO: 2 or at a position corresponding thereto (preferably or generally, asparagine);
(B-1_B-15) substitutions of isoleucine for an amino acid at a position 257 of the amino acid sequence set forth in SEQ ID NO: 2 or at a position corresponding thereto (preferably or generally, leucine), and isoleucine for an amino acid at a position 110 of the amino acid sequence set forth in SEQ ID NO: 2 or at a position corresponding thereto (preferably or generally, valine);
(B-1_B-16) substitutions of isoleucine for an amino acid at a position 257 of the amino acid sequence set forth in SEQ ID NO: 2 or at a position corresponding thereto (preferably or generally, leucine), and methionine for an amino acid at a position 110 of the amino acid sequence set forth in SEQ ID NO: 2 or at a position corresponding thereto (preferably or generally, valine);
(B-1_B-17) substitutions of isoleucine for an amino acid at a position 257 of the amino acid sequence set forth in SEQ ID NO: 2 or at a position corresponding thereto (preferably or generally, leucine), and leucine for an amino acid at a position 110 of the amino acid sequence set forth in SEQ ID NO: 2 or at a position corresponding thereto (preferably or generally, valine);
(B-1_B-18) substitutions of isoleucine for an amino acid at a position 257 of the amino acid sequence set forth in SEQ ID NO: 2 or at a position corresponding thereto (preferably or generally, leucine), and phenylalanine for an amino acid at a position 110 of the amino acid sequence set forth in SEQ ID NO: 2 or at a position corresponding thereto (preferably or generally, valine); and
(B-1_B-19) substitutions of isoleucine for an amino acid at a position 257 of the amino acid sequence set forth in SEQ ID NO: 2 or at a position corresponding thereto (preferably or generally, leucine), and isoleucine for an amino acid at a position 118 of the amino acid sequence set forth in SEQ ID NO: 2 or at a position corresponding thereto (preferably or generally, cysteine).
<12> The method described in any one of the above items <1> to <11>, wherein the protein (D) is a protein having NADH-type FabG activity.
<13> The method described in any one of the above items <1> to <12>, wherein the protein (D) is a protein consisting of an amino acid sequence in which 1 or several amino acids, preferably 1 or more and 98 or less amino acids, more preferably 1 or more and 86 or less amino acids, further preferably 1 or more and 73 or less amino acids, furthermore preferably 1 or more and 61 or less amino acids, furthermore preferably 1 or more and 49 or less amino acids, furthermore preferably 1 or more and 36 or less amino acids, furthermore preferably 1 or more and 24 or less amino acids, furthermore preferably 1 or more and 17 or less amino acids, furthermore preferably 1 or more and 12 or less amino acids, furthermore preferably 1 or more and 9 or less amino acids, furthermore preferably 1 or more and 7 or less amino acids, furthermore preferably 1 or more and 4 or less amino acids, and furthermore preferably 1 or 2 amino acids, are deleted, substituted, inserted or added to the amino acid sequence of the protein (C), and having FabG activity.
<14> The method described in any one of the above items <1> to <13>, wherein the gene encoding the protein (A) or (B), and the gene encoding the protein (C) or (D) are a gene consisting of the following DNA (a) or (b), and a gene consisting of the following DNA (c) or (d):
(a) a DNA consisting of the nucleotide sequence set forth in SEQ ID NO: 1;
(b) a DNA consisting of a nucleotide sequence having 60% or more, preferably 70% or more, more preferably 75% or more, further preferably 80% or more, furthermore preferably 85% or more, furthermore preferably 90% or more, furthermore preferably 93% or more, furthermore preferably 95% or more, furthermore preferably 96% or more, furthermore preferably 97% or more, furthermore preferably 98% or more, and furthermore preferably 99% or more, identity with the nucleotide sequence set forth in SEQ ID NO: 1 and encoding a protein having TE activity;
(c) a DNA consisting of the nucleotide sequence set forth in SEQ ID NO: 3; and
(d) a DNA consisting of a nucleotide sequence having 60% or more, preferably 70% or more, more preferably 75% or more, further preferably 80% or more, furthermore preferably 85% or more, furthermore preferably 90% or more, furthermore preferably 93% or more, furthermore preferably 95% or more, furthermore preferably 96% or more, furthermore preferably 97% or more, furthermore preferably 98% or more, and furthermore preferably 99% or more, identity with the nucleotide sequence set forth in SEQ ID NO: 3 and encoding a protein having FabG activity.
<15> The method described in the above item <14>, wherein the DNA (b) is a DNA consisting of a nucleotide sequence in which 1 or several nucleotides, preferably 1 or more and 427 or less nucleotides, more preferably 1 or more and 373 or less nucleotides, further preferably 1 or more and 320 or less nucleotides, furthermore preferably 1 or more and 267 or less nucleotides, furthermore preferably 1 or more and 213 or less nucleotides, furthermore preferably 1 or more and 160 or less nucleotides, furthermore preferably 1 or more and 106 or less nucleotides, furthermore preferably 1 or more and 74 or less nucleotides, furthermore preferably 1 or more and 53 or less nucleotides, furthermore preferably 1 or more and 42 or less nucleotides, furthermore preferably 1 or more and 32 or less nucleotides, furthermore preferably 1 or more and 21 or less nucleotides, and furthermore preferably 1 or more and 10 or less nucleotides, are deleted, substituted, inserted or added to the nucleotide sequence of the DNA (a), and encoding the protein (A) or (B) having TE activity, or a DNA capable of hybridizing with a DNA consisting of a nucleotide sequence complementary with the DNA (a) under a stringent condition, and encoding the protein (A) or (B) having TE activity.
<16> The method described in the above item <14> or <15>, wherein the DNA (b) is a DNA consisting of a nucleotide sequence having at least one nucleotide substitution selected from the group consisting of the following (b-1) to (b-11), preferably the nucleotide substitution of the (b-1), and encoding the protein (A) or (B):
(b-1) substitution of nucleotides encoding isoleucine for nucleotides at positions 769 to 771 of the nucleotide sequence set forth in SEQ ID NO: 1, or at positions corresponding thereto;
(b-2) substitution of nucleotides encoding arginine for nucleotides at positions 751 to 753 of the nucleotide sequence set forth in SEQ ID NO: 1, or at positions corresponding thereto;
(b-3) substitution of nucleotides encoding lysine for nucleotides at positions 751 to 753 of the nucleotide sequence set forth in SEQ ID NO: 1, or at positions corresponding thereto;
(b-4) substitution of nucleotides encoding histidine for nucleotides at positions 751 to 753 of the nucleotide sequence set forth in SEQ ID NO: 1, or at positions corresponding thereto;
(b-5) substitution of nucleotides encoding isoleucine for nucleotides at positions 760 to 762 of the nucleotide sequence set forth in SEQ ID NO: 1, or at positions corresponding thereto;
(b-6) substitution of nucleotides encoding tyrosine for nucleotides at positions 760 to 762 of the nucleotide sequence set forth in SEQ ID NO: 1, or at positions corresponding thereto;
(b-7) substitution of nucleotides encoding methionine for nucleotides at positions 769 to 771 of the nucleotide sequence set forth in SEQ ID NO: 1, or at positions corresponding thereto;
(b-8) substitution of nucleotides encoding valine for nucleotides at positions 769 to 771 of the nucleotide sequence set forth in SEQ ID NO: 1, or at positions corresponding thereto;
(b-9) substitution of nucleotides encoding phenylalanine for nucleotides at positions 769 to 771 of the nucleotide sequence set forth in SEQ ID NO: 1, or at positions corresponding thereto;
(b-10) substitution of nucleotides encoding cysteine for nucleotides at positions 796 to 798 of the nucleotide sequence set forth in SEQ ID NO: 1, or at positions corresponding thereto; and
(b-11) substitution of nucleotides encoding tyrosine for nucleotides at positions 811 to 813 of the nucleotide sequence set forth in SEQ ID NO: 1, or at positions corresponding thereto.
<17> The method described in the above item <16>, wherein the DNA (b) is a DNA consisting of a nucleotide sequence having at least one nucleotide substitution selected from the group consisting of the following (b-12) to (b-19), in addition to at least one nucleotide substitution selected from the group consisting of the (b-1) to (b-11), and encoding the protein (A) or (B):
(b-12) substitution of nucleotides encoding isoleucine for nucleotides at positions 316 to 318 of the nucleotide sequence set forth in SEQ ID NO: 1, or at positions corresponding thereto;
(b-13) substitution of nucleotides encoding lysine for nucleotides at positions 322 to 324 of the nucleotide sequence set forth in SEQ ID NO: 1, or at positions corresponding thereto;
(b-14) substitution of nucleotides encoding arginine for nucleotides at positions 322 to 324 of the nucleotide sequence set forth in SEQ ID NO: 1, or at positions corresponding thereto;
(b-15) substitution of nucleotides encoding isoleucine for nucleotides at positions 328 to 330 of the nucleotide sequence set forth in SEQ ID NO: 1, or at positions corresponding thereto;
(b-16) substitution of nucleotides encoding methionine for nucleotides at positions 328 to 330 of the nucleotide sequence set forth in SEQ ID NO: 1, or at positions corresponding thereto;
(b-17) substitution of nucleotides encoding leucine for nucleotides at positions 328 to 330 of the nucleotide sequence set forth in SEQ ID NO: 1, or at positions corresponding thereto;
(b-18) substitution of nucleotides encoding phenylalanine for nucleotides at positions 328 to 330 of the nucleotide sequence set forth in SEQ ID NO: 1, or at positions corresponding thereto; and
(b-19) substitution of nucleotides encoding isoleucine for nucleotides at positions 352 to 354 of the nucleotide sequence set forth in SEQ ID NO: 1, or at positions corresponding thereto.
<18> The method described in any one of the above items <14> to <17>, wherein the DNA (b) is a DNA consisting of a nucleotide sequence having at least one nucleotide substitution selected from the group consisting of the following (b-1_b-12) to (b-1_b-19), and encoding the protein (A) or (B):
(b-1_b-12) substitutions of nucleotides encoding isoleucine for nucleotides at positions 769 to 771 of the nucleotide sequence set forth in SEQ ID NO: 1 or at positions corresponding thereto, and nucleotides encoding isoleucine for nucleotides at positions 316 to 318 of the nucleotide sequence set forth in SEQ ID NO: 1 or at positions corresponding thereto;
(b-1_b-13) substitutions of nucleotides encoding isoleucine for nucleotides at positions 769 to 771 of the nucleotide sequence set forth in SEQ ID NO: 1 or at positions corresponding thereto, and nucleotides encoding lysine for nucleotides at positions 322 to 324 of the nucleotide sequence set forth in SEQ ID NO: 1 or at positions corresponding thereto;
(b-1_b-14) substitutions of nucleotides encoding isoleucine for nucleotides at positions 769 to 771 of the nucleotide sequence set forth in SEQ ID NO: 1 or at positions corresponding thereto, and nucleotides encoding arginine for nucleotides at positions 322 to 324 of the nucleotide sequence set forth in SEQ ID NO: 1 or at positions corresponding thereto;
(b-1_b-15) substitutions of nucleotides encoding isoleucine for nucleotides at positions 769 to 771 of the nucleotide sequence set forth in SEQ ID NO: 1 or at positions corresponding thereto, and nucleotides encoding isoleucine for nucleotides at positions 328 to 330 of the nucleotide sequence set forth in SEQ ID NO: 1 or at positions corresponding thereto;
(b-1_b-16) substitutions of nucleotides encoding isoleucine for nucleotides at positions 769 to 771 of the nucleotide sequence set forth in SEQ ID NO: 1 or at positions corresponding thereto, and nucleotides encoding methionine for nucleotides at positions 328 to 330 of the nucleotide sequence set forth in SEQ ID NO: 1 or nucleotides at positions corresponding thereto;
(b-1_b-17) substitutions of nucleotides encoding isoleucine for nucleotides at positions 769 to 771 of the nucleotide sequence set forth in SEQ ID NO: 1 or at positions corresponding thereto, and nucleotides encoding leucine for nucleotides at positions 328 to 330 of the nucleotide sequence set forth in SEQ ID NO: 1 or at positions corresponding thereto;
(b-1_b-18) substitutions of nucleotides encoding isoleucine for nucleotides at positions 769 to 771 of the nucleotide sequence set forth in SEQ ID NO: 1 or at positions corresponding thereto, and nucleotides encoding phenylalanine for nucleotides at positions 328 to 330 of the nucleotide sequence set forth in SEQ ID NO: 1 or at positions corresponding thereto; and
(b-1_b-19) substitutions of nucleotides encoding isoleucine for nucleotides at positions 769 to 771 of the nucleotide sequence set forth in SEQ ID NO: 1 or at positions corresponding thereto, and nucleotides encoding isoleucine for nucleotides at positions 352 to 354 of the nucleotide sequence set forth in SEQ ID NO: 1 or at positions corresponding thereto.
<19> The method described in any one of the above items <14> to <18>, wherein the DNA (d) is a DNA encoding a protein having NADH-type FabG activity.
<20> The method described in any one of the above items <14> to <19, wherein the DNA (d) is a DNA consisting of a nucleotide sequence in which 1 or several nucleotides, preferably 1 or more and 296 or less nucleotides, more preferably 1 or more and 259 or less nucleotides, further preferably 1 or more and 222 or less nucleotides, furthermore preferably 1 or more and 185 or less nucleotides, furthermore preferably 1 or more and 148 or less nucleotides, furthermore preferably 1 or more and 111 or less nucleotides, furthermore preferably 1 or more and 74 or less nucleotides, furthermore preferably 1 or more and 51 or less nucleotides, furthermore preferably 1 or more and 37 or less nucleotides, furthermore preferably 1 or more and 29 or less nucleotides, furthermore preferably 1 or more and 22 or less nucleotides, furthermore preferably 1 or more and 14 or less nucleotides, and furthermore preferably 1 or more and 7 or less nucleotides, are deleted, substituted, inserted or added to the nucleotide sequence of the DNA (c), and encoding the protein (C) or (D) having FabG activity, or a DNA capable of hybridizing with a DNA consisting of a nucleotide sequence complementary with the DNA (c) under a stringent condition, and encoding the protein (C) or (D) having FabG activity.
<21> The method described in any one of the above items <1> to <20>, wherein the host or the transformant is a microorganism or a transformant of microorganism.
<22> The method described in the above item <21>, wherein the microorganism is a microorganism having only a NADPH-type FabG as a FabG.
<23> The method described in the above item <22>, wherein the microorganism is a microorganism selected from Escherichia coli, Pseudomonas aeruginosa and Bacillus subtilis, preferably is Escherichia coli.
<24> The method described in any one of the above items <1> to <23>, wherein the medium-chain fatty acids or the lipids containing the same as components are fatty acids having 8 or more and 10 or less carbon atoms or lipids containing the same as components, more preferably fatty acids having 8 or 10 carbon atoms or lipids containing the same as components, further preferably saturated fatty acids having 8 or 10 carbon atoms (caprylic acid or capric acid) or lipids containing the same as components, and furthermore preferably saturated fatty acids having 8 carbon atoms (caprylic acid) or lipids containing the same as components.
<25> The method described in any one of the above items <1> to <24>, wherein the lipids contain a fatty acid or a fatty acid ester compound thereof, preferably a medium-chain fatty acid or a fatty acid ester compound thereof, more preferably a fatty acid having 8 or more and 10 or less carbon atoms or a fatty acid ester compound thereof, further preferably a fatty acid having 8 or 10 carbon atoms or a fatty acid ester compound thereof, furthermore preferably a saturated fatty acid having 8 or 10 carbon atoms or a fatty acid ester compound thereof, and furthermore preferably a saturated fatty acid having 8 carbon atoms or a fatty acid ester compound thereof.
<26> A transformant, wherein expression of a gene encoding the protein (A) or (B), and expression of a gene encoding the protein (C) or (D) is enhanced in a host cell.
<27> A transformant, wherein a gene encoding the protein (A) or (B) and a gene encoding the protein (C) or (D), or a recombinant vector containing the same is introduced into a host.
<28> A method of producing a transformant, containing introducing a gene encoding the protein (A) or (B) and a gene encoding the protein (C) or (D), or a recombinant vector containing the same into a host.
<29> The transformant or the method of preparing the same described in any one of the above items <26> to <28>, wherein the protein (B) is a protein specified in any one of the above items <8> to <11>.
<30> The transformant or the method of preparing the same described in any one of the above items <26> to <29>, wherein the protein (D) is a protein specified in the above item <12> or <13>.
<31> The transformant or the method of preparing the same described in any one of the above items <26> to <30>, wherein a gene encoding the protein (A) or (B), and a gene encoding the protein (C) or (D) are a gene consisting of the DNA (a) or (b), and a gene consisting of the DNA (c) or (d) respectively.
<32> The transformant or the method of preparing the same described in the above item <31>, wherein the DNA (b) is a DNA specified in any one of the above items <15> to <18>.
<33> The transformant or the method of preparing the same described in the above item <31>, wherein the DNA (d) is a DNA specified in the above item <19> or <20>.
<34> The transformant or the method of preparing the same described in any one of the above items <26> to <33>, wherein the host or the transformant is a microorganism or a transformant of a microorganism.
<35> The transformant or the method of preparing the same described in the above item <34>, wherein the microorganism is a microorganism having only a NADPH-type FabG as a FabG.
<36> The transformant or the method of preparing the same described in the above item <35>, wherein the microorganism is a microorganism selected from Escherichia coli, Pseudomonas aeruginosa and Bacillus subtilis, preferably is Escherichia coli.
<37> Use of the transformant, or a transformant obtained by the method of preparing the same described in any one of the above items <26> to <36>, for producing lipids.
<38> The use described in the above item <37>, wherein the lipids contain a fatty acid or a fatty acid ester compound thereof, preferably a medium-chain fatty acid or a fatty acid ester compound thereof, more preferably a fatty acid having 8 or more and 10 or less carbon atoms or a fatty acid ester compound thereof, further preferably a fatty acid having 8 or 10 carbon atoms or a fatty acid ester compound thereof, furthermore preferably a saturated fatty acid having 8 or 10 carbon atoms or a fatty acid ester compound thereof, and furthermore preferably a saturated fatty acid having 8 carbon atoms or a fatty acid ester compound thereof.

### EXAMPLES

Hereinafter, the present invention will be described more in detail with reference to Examples, but the present invention is not limited thereto. Herein, the nucleotide sequences of the primers used in Examples are shown in Table 1.

TABLE 1

| Primer Name | Nucleotide Sequence (5′→3′) | SEQ ID NO: |
|---|---|---|
| pBS-F | GCGTTAATATTTTGTTAAAATTCGC | SEQ ID NO: 7 |
| pBS-R | AGCTGTTTCCTGTGTGAAATTG | SEQ ID NO: 8 |
| pBS/CpTE-F | ACACAGGAAACAGCTATGGCTAACGGTTCTGCAGTAAC | SEQ ID NO: 9 |
| CpTE/pBS-R | ACAAAATATTAACGCTCAAGTCTTTCCTGTTGATATCGCC | SEQ ID NO: 10 |
| CpTE/RBS-R | GGTCTGCCTCCTGTTCAAGTCTTTCCTGTTGATATCGCC | SEQ ID NO: 11 |
| RBS/CtfabG-F | AACAGGAGGCAGACCATGAAACTGCAGGGTCGGGTTG | SEQ ID NO: 12 |
| CtfabG/pBS-R | ACAAAATATTAACGCTCAGAGCGACATGCCGCCGCTG | SEQ ID NO: 13 |
| RBS/EcfabG-F | AACAGGAGGCAGACCATGAATTTTGAAGGAAAAATCGCACTGG | SEQ ID NO: 14 |
| EcfabG/pBS-R | ACAAAATATTAACGCTCAGACCATGTACATCCCGC | SEQ ID NO: 15 |

### Example 1 Lipid Production by Escherichia coli into which the CpTE Gene is Introduced

### (1) Construction of Plasmid for CpTE Gene Expression

By using the pBS-SK(−) plasmid (manufactured by Agilent Technologies) as a template, and the primer pBS-F (SEQ ID NO: 7) and the primer pBS-R (SEQ ID NO: 8) shown in Table 1, PCR was carried out to amplify a linearized DNA sequence of the pBS-SK(−).

Further, TE gene derived from Cuphea palustris (GenBank: 038188.1) was artificially synthesized. Using thus-synthesized DNA sequence as a template, and the primer pBS/CpTE-F (SEQ ID NO: 9) and the primer CpTE/pBS-R (SEQ ID NO: 10) shown in Table 1, PCR was carried out to amplify a DNA fragment of CpTE gene wherein a sequence of a putative chloroplast transit signal was deleted.

Then, the linearized DNA sequence of the pBS-SK(−) and the DNA fragment of the CpTE gene were mixed to carry out cloning by In-Fusion (registered trademark) PCR cloning method (Clontech), and thereby pBS-CpTE plasmid in which the CpTE gene was inserted at downstream of lacO promoter of the pBS-SK(−) plasmid was obtained.

### (2) Construction of Plasmid for CpTE-CtFabG Gene Expression

By using the pBS-CpTE plasmid as a template, and primer pairs of the primer pBS-F (SEQ ID NO: 7) and the primer CpTE/RBS-R (SEQ ID NO: 11) shown in Table 1, PCR was carried out to amplify a linearized pBS-CpTE plasmid.

CtFabG gene (UniProt (www.uniprot.org/): RALTA_A2639) was artificially synthesized. Using thus-synthesized DNA sequence as a template, and primer pairs of the primer RBS/CtfabG-F (SEQ ID NO: 12) and the primer CtfabG/pBS-R (SEQ ID NO: 13) shown in Table 1, PCR was carried out to amplify a DNA fragment of CtFabG gene.

The linearized DNA sequence of the pBS-CpTE and the DNA fragment of the CtFabG gene were mixed to carry out cloning by In-Fusion (registered trademark) PCR cloning method (Clontech), and thereby pBS-CpTE-CtFabG plasmid (plasmid for CpTE-CtFabG gene expression) in which the CpTE gene and CtFabG gene were inserted at downstream of lacO promoter of the pBS-SK(−) plasmid was obtained.

(3) Construction of Plasmid for CpTE-FabG, Derived from Escherichia coli, Gene Expression

By using the pBS-CpTE plasmid as a template, and primer pairs of the primer pBS-F (SEQ ID NO: 7) and the primer CpTE/RBS-R (SEQ ID NO: 11) shown in Table 1, PCR was carried out to amplify a linearized pBS-CpTE plasmid.

Further, using genomic DNA extracted from Escherichia coli as a template, and primer pairs of the primer RBS/EcfabG-F (SEQ ID NO: 14) and the primer EcfabG/pBS-R (SEQ ID NO: 15) shown in Table 1, PCR was carried out to obtain a DNA fragment of a gene (hereinafter, also referred to as “EcFabG gene”) encoding FabG derived from Escherichia coli (hereinafter, also referred to as “EcFabG”).

Then, the linearized DNA sequence of the pBS-CpTE and the DNA fragment of the EcFabG gene were mixed to carry out cloning by In-Fusion (registered trademark) PCR cloning method (Clontech), and thereby pBS-CpTE-EcFabG plasmid (plasmid for CpTE-EcFabG gene expression) in which the CpTE gene and the EcFabG gene were inserted at downstream of lacO promoter of the pBS-SK(−) plasmid was obtained.

(4) Introduction of Plasmid for Gene Expression into Escherichia coli, and Lipid Production Using Thus-Obtained Transformant

An Escherichia coli mutant strain K27 (fadD88) (Overath et al, Eur. J. Biochem. 7, 559-574, 1969) was transformed by a competent cell transformation method, using the plasmid for CpTE gene expression, and the plasmid for CpTE-CtFabG gene expression or the plasmid for CpTE-EcFabG gene expression.

The transformed strain K27 was stand overnight at 30° C., and a colony thus obtained was inoculated in 1 mL of LBAmp liquid medium (Bacto Trypton 1%, Yeast Extract 0.5%, NaCl 1%, and Ampicillin sodium 50 μg/mL), and then cultured overnight at 30° C. The culture fluid of 2 μL was inoculated to 2 mL of Overnight Express Instant TB Medium (Novagen) with 1% of glycerol, and was subjected to shaking culture at 30° C. After 24 hours cultivation, lipid components contained in the culture fluid were analyzed by the method described below.

(4) Extraction of Lipid from Escherichia coli Culture Fluid and Analysis of Fatty Acids Contained Therein

To 0.5 mL of the culture fluid, 25 μL of 1 mg/mL 7-pentadecanone as an internal standard was added, and then 10 μL of 2 N hydrochloric acid and 2 mL of hexane were further added. The mixture was vigorously stirred and centrifuged for 10 minutes at 3,000 rpm. Then the hexane layer (upper layer) was collected with pasteur pipette into a test tube with screw cap. A nitrogen gas was blown onto the resultant hexane layer to be dried into solid, then 1 mL of 14% solution of boron trifluoride (manufactured by Sigma-Aldrich) was added to the sample, and the mixture was kept warm at 80° C. for 30 minutes. Thereafter, 1 mL of saturated saline and 1 mL of hexane were added thereto, and the mixture was vigorously stirred and then was left for 30 minutes at room temperature. Then, the hexane layer being upper layer was collected to obtain fatty acid esters.

The obtained fatty acid esters were provided for gas chromatographic analysis. Using 7890A (Agilent Technologies), gas chromatographic analysis was performed under the conditions as follows.

(Analysis conditions)
Capillary column: DB-1 MS (30 m×200 μm×0.25 μm, manufactured by J&W Scientific)
Mobile phase: high purity helium
Flow rate inside the column: 1.0 mL/min
Temperature rise program: maintained for 1 minute at 70° C.→70 to 200° C. (temperature increase at 20° C./minute)→200 to 320° C. (temperature increase at 50° C./minute)→maintained for 5 minutes at 320° C.
Equilibration time: 1 min
Injection port: split injection (split ratio: 100:1)
Pressure: 14.49 psi, 104 mL/min
Amount of injection: 1 μL
Cleaning vial: methanol/chloroform
Detector temperature: 300° C.

The fatty acid esters were identified by providing the identical sample for gas chromatography—mass spectrometry analysis under identical conditions described above.

Amounts of the fatty acid methyl esters were quantitatively determined based on the peak areas of waveform data obtained by the above gas chromatographic analysis. The peak area was compared with that of 7-pentadecanone as the internal standard, and corrections between the samples were carried out, and then the amount of each of the fatty acids and the total amount thereof per liter of the culture fluid were calculated.

Tables 2 and 3 show the results. In addition, the results in Tables 2 and 3 are shown in terms of an average value of the results of independent culture three times and chromatography analyses thereof.

Herein, in Tables below, C8 means a C8:0 fatty acid, 010 means a sum of C10:0 and C10:1 fatty acids, C12 means a sum of C12:0 and C12:1 fatty acids, C14 means a sum of 014:0 and C14:1 fatty acids, C16 means a sum of C16:0, C16:1, C16:2 and C16:3 fatty acids, and C18 means a sum of C18:0, C18:1, C18:2, C18:3, C18:4 and C18:5 fatty acids. Further, in Tables below, “Total production amount of fatty acids” (total amount of fatty acids produced) means a sum of these fatty acids.

TABLE 2
(N = 3)

| | | | | | | | Total production |
|---|---|---|---|---|---|---|---|
| | Production amount of fatty acids (mg/L) | | | | | | amount of fatty acids |
| | C8 | C10 | C12 | C14 | C16 | C18 | (mg/L) |
| CpTE | 36.8 ± 6.9 | 1.5 ± 0.2 | 0 ± 0 | 0 ± 0 | 0 ± 0 | 0 ± 0 | 38.3 ± 7.0 |
| CpTE + | 124.4 ± 14.6 | 8.3 ± 0.7 | 2.6 ± 0.3 | 1.6 ± 0.1 | 4.7 ± 0.2 | 0 ± 0 | 141.7 ± 15.6 |
| CtFabG | | | | | | | |

TABLE 3
(N = 3)

| | | | | | | | Total production |
|---|---|---|---|---|---|---|---|
| | Production amount of fatty acids (mg/L) | | | | | | amount of fatty acids |
| | C8 | C10 | C12 | C14 | C16 | C18 | (mg/L) |
| CpTE | 38.0 ± 4.5 | 0.7 ± 1.2 | 0 ± 0 | 0 ± 0 | 0 ± 0 | 0 ± 0 | 38.7 ± 5.6 |
| CpTE + | 42.2 ± 8.7 | 1.4 ± 1.3 | 0 ± 0 | 0 ± 0 | 0 ± 0 | 0 ± 0 | 43.6 ± 9.9 |
| EcFabG | | | | | | | |

As is apparent from Table 2, production amount of C8 fatty acid and total amount of fatty acids produced were highly increased in the transformant into which pBS-CpTE-CtFabG plasmid was introduced to introduce the CpTE gene and CtFabG gene, in comparison with that in the transformant into which only CpTE gene was introduced. Specifically, amount of C8 fatty acid was improved by 3.38 times and total amount of fatty acids produced was improved by 3.70 times in the transformant wherein the CpTE gene and CtFabG gene were introduced into an Escherichia coli, in comparison with those in the transformant wherein only the CpTE gene was introduced into an Escherichia coli.

In contrast, as apparent from Table 3, there was no significant difference in production amount of C8 fatty acid and total amount of fatty acids produced between the transformant wherein pBS-CpTE-EcFabG plasmid was introduced into an Escherichia coli to introduce the CpTE gene and EcFabG gene, and the transformant wherein only the CpTE gene was introduced into an Escherichia coli.

As described above, the transformant in which productivity of medium-chain fatty acids has been significantly improved can be prepared by introducing the FabG gene specified in the present invention, in addition to the TE gene, into a host cell. Then, productivity of medium-chain fatty acids and total amount of fatty acids produced can be improved by culturing this transformant.

Having described our invention as related to the present embodiments, it is our intention that the invention not be limited by any of the details of the description, unless otherwise specified, but rather be construed broadly within its spirit and scope.

This application claims priority on Patent Application No. 62/659,793 filed in the US on Apr. 19, 2018, which is entirely herein incorporated by reference.

What is claimed is:

1. A method of producing lipids, comprising the steps of:
culturing a transformant wherein a gene encoding the following protein (A) or (B), and a gene encoding the following protein (C) or (D) are introduced into a host cell, and
producing medium-chain fatty acids or the lipids containing the same as components:
(A) a protein consisting of the amino acid sequence set forth in SEQ ID NO: 2;
(B) a protein consisting of an amino acid sequence having 60% or more identity with the amino acid sequence set forth in SEQ ID NO: 2, and having acyl-ACP thioesterase activity;
(C) a protein consisting of the amino acid sequence set forth in SEQ ID NO: 4; and
(D) a protein consisting of an amino acid sequence having 60% or more identity with the amino acid sequence set forth in SEQ ID NO: 4, and having β-ketoacyl-ACP reductase activity.

2. (canceled)

3. A method of improving productivity of medium-chain fatty acids, comprising the steps of:
culturing a transformant wherein a gene encoding the following protein (A) or (B), and a gene encoding the following protein (C) or (D) are introduced into a host cell, and
improving the productivity of medium-chain fatty acids or lipids containing the same as components:
(A) a protein consisting of the amino acid sequence set forth in SEQ ID NO: 2;
(B) a protein consisting of an amino acid sequence having 60% or more identity with the amino acid sequence set forth in SEQ ID NO: 2, and having acyl-ACP thioesterase activity;
(C) a protein consisting of the amino acid sequence set forth in SEQ ID NO: 4; and
(D) a protein consisting of an amino acid sequence having 60% or more identity with the amino acid sequence set forth in SEQ ID NO: 4, and having β-ketoacyl-ACP reductase activity.

4. The method of claim 1, wherein the protein (B) is a protein consisting of an amino acid sequence having at least one amino acid substitution selected from the group consisting of the following (B-1) to (B-11):
(B-1) substitution of isoleucine for an amino acid at a position corresponding to position 257 of the amino acid sequence set forth in SEQ ID NO: 2;
(B-2) substitution of arginine for an amino acid at a position corresponding to position 251 of the amino acid sequence set forth in SEQ ID NO: 2;
(B-3) substitution of lysine for an amino acid at a position corresponding to position 251 of the amino acid sequence set forth in SEQ ID NO: 2;
(B-4) substitution of histidine for an amino acid at a position corresponding to position 251 of the amino acid sequence set forth in SEQ ID NO: 2;
(B-5) substitution of isoleucine for an amino acid at a position corresponding to position 254 of the amino acid sequence set forth in SEQ ID NO: 2;
(B-6) substitution of tyrosine for an amino acid at a position corresponding to position 254 of the amino acid sequence set forth in SEQ ID NO: 2;
(B-7) substitution of methionine for an amino acid at a position corresponding to position 257 of the amino acid sequence set forth in SEQ ID NO: 2;
(B-8) substitution of valine for an amino acid at a position corresponding to position 257 of the amino acid sequence set forth in SEQ ID NO: 2;
(B-9) substitution of phenylalanine for an amino acid at a position corresponding to position 257 of the amino acid sequence set forth in SEQ ID NO: 2;
(B-10) substitution of cysteine for an amino acid at a position corresponding to position 266 of the amino acid sequence set forth in SEQ ID NO: 2; and
(B-11) substitution of tyrosine for an amino acid at a position corresponding to position 271 of the amino acid sequence set forth in SEQ ID NO: 2.

5. The method of claim 4, wherein the protein (B) is a protein consisting of an amino acid sequence further having at least one amino acid substitution selected from the group consisting of the following (B-12) to (B-19):
(B-12) substitution of isoleucine for an amino acid at a position corresponding to position 106 of the amino acid sequence set forth in SEQ ID NO: 2;
(B-13) substitution of lysine for an amino acid at a position corresponding to position 108 of the amino acid sequence set forth in SEQ ID NO: 2;
(B-14) substitution of arginine for an amino acid at a position corresponding to position 108 of the amino acid sequence set forth in SEQ ID NO: 2;
(B-15) substitution of isoleucine for an amino acid at a position corresponding to position 110 of the amino acid sequence set forth in SEQ ID NO: 2;
(B-16) substitution of methionine for an amino acid at a position corresponding to position 110 of the amino acid sequence set forth in SEQ ID NO: 2;
(B-17) substitution of leucine for an amino acid at a position corresponding to position 110 of the amino acid sequence set forth in SEQ ID NO: 2;
(B-18) substitution of phenylalanine for an amino acid at a position corresponding to position 110 of the amino acid sequence set forth in SEQ ID NO: 2; and
(B-19) substitution of isoleucine for an amino acid at a position corresponding to position 118 of the amino acid sequence set forth in SEQ ID NO: 2.

6. The method of claim 1,
wherein the protein (D) is a protein having NADH-type β-ketoacyl-ACP reductase activity.

7. The method of claim 1,
wherein the host is a microorganism.

8. The method of claim 7, wherein the microorganism is Escherichia coli.

9. The method of claim 1, wherein the lipids contain a saturated fatty acid having 8 carbon atoms or a fatty acid ester compound thereof.

10. A transformant, wherein expression of a gene encoding the following protein (A) or (B), and expression of a gene encoding the following protein (C) or (D) is enhanced in a host cell:
(A) a protein consisting of the amino acid sequence set forth in SEQ ID NO: 2;
(B) a protein consisting of an amino acid sequence having 60% or more identity with the amino acid sequence set forth in SEQ ID NO: 2, and having acyl-ACP thioesterase activity;
(C) a protein consisting of the amino acid sequence set forth in SEQ ID NO: 4; and
(D) a protein consisting of an amino acid sequence having 60% or more identity with the amino acid sequence set forth in SEQ ID NO: 4, and having β-ketoacyl-ACP reductase activity.

11. The transformant of claim 10, wherein the protein (B) is a protein consisting of an amino acid sequence having at least one amino acid substitution selected from the group consisting of the following (B-1) to (B-11):
(B-1) substitution of isoleucine for an amino acid at a position corresponding to position 257 of the amino acid sequence set forth in SEQ ID NO: 2;
(B-2) substitution of arginine for an amino acid at a position corresponding to position 251 of the amino acid sequence set forth in SEQ ID NO: 2;
(B-3) substitution of lysine for an amino acid at a position corresponding to position 251 of the amino acid sequence set forth in SEQ ID NO: 2;
(B-4) substitution of histidine for an amino acid at a position corresponding to position 251 of the amino acid sequence set forth in SEQ ID NO: 2;
(B-5) substitution of isoleucine for an amino acid at a position corresponding to position 254 of the amino acid sequence set forth in SEQ ID NO: 2;
(B-6) substitution of tyrosine for an amino acid at a position corresponding to position 254 of the amino acid sequence set forth in SEQ ID NO: 2;
(B-7) substitution of methionine for an amino acid at a position corresponding to position 257 of the amino acid sequence set forth in SEQ ID NO: 2;
(B-8) substitution of valine for an amino acid at a position corresponding to position 257 of the amino acid sequence set forth in SEQ ID NO: 2;
(B-9) substitution of phenylalanine for an amino acid at a position corresponding to position 257 of the amino acid sequence set forth in SEQ ID NO: 2;
(B-10) substitution of cysteine for an amino acid at a position corresponding to position 266 of the amino acid sequence set forth in SEQ ID NO: 2; and
(B-11) substitution of tyrosine for an amino acid at a position corresponding to position 271 of the amino acid sequence set forth in SEQ ID NO: 2.

12. The transformant of claim 11, wherein the protein (B) is a protein consisting of an amino acid sequence further having at least one amino acid substitution selected from the group consisting of the following (B-12) to (B-19):
(B-12) substitution of isoleucine for an amino acid at a position corresponding to position 106 of the amino acid sequence set forth in SEQ ID NO: 2;
(B-13) substitution of lysine for an amino acid at a position corresponding to position 108 of the amino acid sequence set forth in SEQ ID NO: 2;
(B-14) substitution of arginine for an amino acid at a position corresponding to position 108 of the amino acid sequence set forth in SEQ ID NO: 2;
(B-15) substitution of isoleucine for an amino acid at a position corresponding to position 110 of the amino acid sequence set forth in SEQ ID NO: 2;
(B-16) substitution of methionine for an amino acid at a position corresponding to position 110 of the amino acid sequence set forth in SEQ ID NO: 2;
(B-17) substitution of leucine for an amino acid at a position corresponding to position 110 of the amino acid sequence set forth in SEQ ID NO: 2;
(B-18) substitution of phenylalanine for an amino acid at a position corresponding to position 110 of the amino acid sequence set forth in SEQ ID NO: 2; and
(B-19) substitution of isoleucine for an amino acid at a position corresponding to position 118 of the amino acid sequence set forth in SEQ ID NO: 2.

13. The transformant of claim 10, wherein the protein (D) is a protein having NADH-type β-ketoacyl-ACP reductase activity.

14. The transformant of claim 10, wherein the host is a microorganism.

15. The method of claim 14, wherein the microorganism is Escherichia coli.

16. The method of claim 3, wherein the protein (B) is a protein consisting of an amino acid sequence having at least one amino acid substitution selected from the group consisting of the following (B-1) to (B-11):
(B-1) substitution of isoleucine for an amino acid at a position corresponding to position 257 of the amino acid sequence set forth in SEQ ID NO: 2;
(B-2) substitution of arginine for an amino acid at a position corresponding to position 251 of the amino acid sequence set forth in SEQ ID NO: 2;
(B-3) substitution of lysine for an amino acid at a position corresponding to position 251 of the amino acid sequence set forth in SEQ ID NO: 2;
(B-4) substitution of histidine for an amino acid at a position corresponding to position 251 of the amino acid sequence set forth in SEQ ID NO: 2;
(B-5) substitution of isoleucine for an amino acid at a position corresponding to position 254 of the amino acid sequence set forth in SEQ ID NO: 2;
(B-6) substitution of tyrosine for an amino acid at a position corresponding to position 254 of the amino acid sequence set forth in SEQ ID NO: 2;
(B-7) substitution of methionine for an amino acid at a position corresponding to position 257 of the amino acid sequence set forth in SEQ ID NO: 2;
(B-8) substitution of valine for an amino acid at a position corresponding to position 257 of the amino acid sequence set forth in SEQ ID NO: 2;
(B-9) substitution of phenylalanine for an amino acid at a position corresponding to position 257 of the amino acid sequence set forth in SEQ ID NO: 2;
(B-10) substitution of cysteine for an amino acid at a position corresponding to position 266 of the amino acid sequence set forth in SEQ ID NO: 2; and
(B-11) substitution of tyrosine for an amino acid at a position corresponding to position 271 of the amino acid sequence set forth in SEQ ID NO: 2.

17. The method of claim 16, wherein the protein (B) is a protein consisting of an amino acid sequence further having at least one amino acid substitution selected from the group consisting of the following (B-12) to (B-19):
(B-12) substitution of isoleucine for an amino acid at a position corresponding to position 106 of the amino acid sequence set forth in SEQ ID NO: 2;
(B-13) substitution of lysine for an amino acid at a position corresponding to position 108 of the amino acid sequence set forth in SEQ ID NO: 2;
(B-14) substitution of arginine for an amino acid at a position corresponding to position 108 of the amino acid sequence set forth in SEQ ID NO: 2;
(B-15) substitution of isoleucine for an amino acid at a position corresponding to position 110 of the amino acid sequence set forth in SEQ ID NO: 2;
(B-16) substitution of methionine for an amino acid at a position corresponding to position 110 of the amino acid sequence set forth in SEQ ID NO: 2;
(B-17) substitution of leucine for an amino acid at a position corresponding to position 110 of the amino acid sequence set forth in SEQ ID NO: 2;
(B-18) substitution of phenylalanine for an amino acid at a position corresponding to position 110 of the amino acid sequence set forth in SEQ ID NO: 2; and
(B-19) substitution of isoleucine for an amino acid at a position corresponding to position 118 of the amino acid sequence set forth in SEQ ID NO: 2.

18. The method of claim 3, wherein the protein (D) is a protein having NADH-type β-ketoacyl-ACP reductase activity.

19. The method of claim 3, wherein the host is a microorganism.

20. The method of claim 19, wherein the microorganism is Escherichia coli.

21. The method of claim 3, wherein the lipids contain a saturated fatty acid having 8 carbon atoms or a fatty acid ester compound thereof.


SEQUENCE LISTING (17 sequences)

SEQ ID NO: 1 - DNA, 1068 bp, Cuphea palustris
atggctaacggttctgcagtaactctaaagtctggcagcctcaacactcaggaggacactttgtcgtcgtcccctcctccccgggctttttttaaccagttgcctgattggagtatgcttctgactgcaatcacaaccgtcttcgtggcaccagagaagcggtggactatgtttgataggaaatctaagaggcctaacatgctcatggactcgtttgggttggagagagttgttcaggatgggctcgtgttcagacagagtttttcgattaggtcttatgaaatatgcgctgatcgaacagcctctatagagacggtgatgaaccacgtccaggaaacatcactcaatcaatgtaagagtataggtcttctcgatgacggctttggtcgtagtcctgagatgtgtaaaagggacctcatttgggtggttacaagaatgaagataatggtgaatcgctatccaacttggggcgatactatcgaggtcagtacctggctctctcaatcggggaaaatcggtatgggtcgcgattggctaataagtgattgcaacacaggagaaattcttgtaagagcaacgagtgtgtatgccatgatgaatcaaaagacgagaagattctcaaaactcccacacgaggttcgccaggaatttgcgcctcattttctggactctcctcctgccattgaagacaacgacggtaaattgcagaagtttgatgtgaagactggtgattccattcgcaagggtctaactccggggtggtatgacttggatgtcaatcagcacgtaagcaacgtgaagtacattgggtggattctcgagagtatgccaacagaagttttggagactcaggagctatgttctctcacccttgaatataggcgggaatgcggaagggacagtgtgctggagtccgtgacctctatggatccctcaaaagttggagaccggtttcagtaccggcaccttctgcggcttgaggatggggctgatatcatgaagggaagaactgagtggcggccgaagaatgcaggaactaacggggcgatatcaacaggaaagacttga

SEQ ID NO: 2 - protein, 355 aa, Cuphea palustris
MANGSAVTLKSGSLNTQEDTLSSSPPPRAFFNQLPDWSMLLTAITTVFVAPEKRWTMFDRKSKRPNMLMDSFGLERVVQDGLVFRQSFSIRSYEICADRTASIETVMNHVQETSLNQCKSIGLLDDGFGRSPEMCKRDLIWVVTRMKIMVNRYPTWGDTIEVSTWLSQSGKIGMGRDWLISDCNTGEILVRATSVYAMMNQKTRRFSKLPHEVRQEFAPHFLDSPPAIEDNDGKLQKFDVKTGDSIRKGLTPGWYDLDVNQHVSNVKYIGWILESMPTEVLETQELCSLTLEYRRECGRDSVLESVTSMDPSKVGDRFQYRHLLRLEDGADIMKGRTEWRPKNAGTNGAISTGKT

SEQ ID NO: 3 - DNA, 741 bp, Cupriavidus taiwanensis
atgaaactgcagggtcgggttgccatcatcaccggtgccgccgccggcatcggctttgccaccgcgcagcgctttgccgaagacggcgccatcgtcgtgctgtgcgacgtacaggaagcgcgcgtgcgagaagccgccgccaggctggcggccaccggcgccaccgtatcggcctaccgcgtcgacgtgacgcgccgtgacgaggtcgacgccatggtcgccgccgtgctggccgcgcaccagcgtgtcgatatcctcgtcaacaatgccggcatcaccaaggacgcgcgcctggccaagatgaccgaggcccagttcgatgccgtcatcgacgtcaacctgaaaggcgtattcaactgcgcgcaggccgtggccgggctgatgaccgagcagggcaagggcgtgatcctgaacgcctcgagcgtggtcggcctgtacggcaatttcggccagaccaactacgccgccagcaaattcggcgtgatcggcttcaccaagacctgggcgcgcgagctgggccccaagggcgtgcgcgtgaacgcggtgtgcccgggcttcgtcaataccgagatcctgcagaccgtgccggacaaggtgctggacggcatgacgtcgtcatgctggctgcgccggctggccgagccggccgagattgccagcatctacgccttcctggccagcgacgacgccagctacgtcaacggcgtggcaatcgaggccagcggcggcatgtcgctctga

SEQ ID NO: 4 - protein, 246 aa, Cupriavidus taiwanensis
MKLQGRVAIITGAAAGIGFATAQRFAEDGAIVVLCDVQEARVREAAARLAATGATVSAYRVDVTRRDEVDAMVAAVLAAHQRVDILVNNAGITKDARLAKMTEAQFDAVIDVNLKGVFNCAQAVAGLMTEQGKGVILNASSVVGLYGNFGQTNYAASKFGVIGFTKTWARELGPKGVRVNAVCPGFVNTEILQTVPDKVLDGMTSSCWLRRLAEPAEIASIYAFLASDDASYVNGVAIEASGGMSL

SEQ ID NO: 5 - DNA, 735 bp, Escherichia coli
atgaattttgaaggaaaaatcgcactggtaaccggtgcaagccgcggaattggccgcgcaattgctgaaacgctcgcagcccgtggcgcgaaagttattggcactgcgaccagtgaaaatggcgctcaggcgatcagtgattatttaggtgccaacggcaaaggtctgatgttgaatgtgaccgacccggcatctatcgaatctgttctggaaaaaattcgcgcagaatttggtgaagtggatatcctggtcaataatgccggtatcactcgtgataacctgttaatgcgaatgaaagatgaagagtggaacgatattatcgaaaccaacctttcatctgttttccgtctgtcaaaagcggtaatgcgcgctatgatgaaaaagcgtcatggtcgtattatcactatcggttctgtggttggtaccatgggaaatggcggtcaggccaactacgctgcggcgaaagcgggcttgatcggcttcagtaaatcactggcgcgcgaagttgcgtcacgcggtattactgtaaacgttgttgctccgggctttattgaaacggacatgacacgtgcgctgagcgatgaccagcgtgcgggtatcctggcgcaggttcctgcgggtcgcctcggcggcgcacaggaaatcgccaacgcggttgcattcctggcatccgacgaagcagcttacatcacgggtgaaactttgcatgtgaacggcgggatgtacatggtctga

SEQ ID NO: 6 - protein, 244 aa, Escherichia coli
MNFEGKIALVTGASRGIGRAIAETLAARGAKVIGTATSENGAQAISDYLGANGKGLMLNVTDPASIESVLEKIRAEFGEVDILVNNAGITRDNLLMRMKDEEWNDIIETNLSSVFRLSKAVMRAMMKKRHGRIITIGSVVGTMGNGGQANYAAAKAGLIGFSKSLAREVASRGITVNVVAPGFIETDMTRALSDDQRAGILAQVPAGRLGGAQEIANAVAFLASDEAAYITGETLHVNGGMYMV

SEQ ID NO: 7 - DNA, 25 bp, Artificial Sequence
  Primer No. 7
gcgttaatattttgttaaaattcgc

SEQ ID NO: 8 - DNA, 22 bp, Artificial Sequence
  Primer No. 8
agctgtttcctgtgtgaaattg

SEQ ID NO: 9 - DNA, 38 bp, Artificial Sequence
  Primer No. 9
acacaggaaacagctatggctaacggttctgcagtaac

SEQ ID NO: 10 - DNA, 40 bp, Artificial Sequence
  Primer No. 10
acaaaatattaacgctcaagtctttcctgttgatatcgcc

SEQ ID NO: 11 - DNA, 39 bp, Artificial Sequence
  Primer No. 11
ggtctgcctcctgttcaagtctttcctgttgatatcgcc

SEQ ID NO: 12 - DNA, 37 bp, Artificial Sequence
  Primer No. 12
aacaggaggcagaccatgaaactgcagggtcgggttg

SEQ ID NO: 13 - DNA, 37 bp, Artificial Sequence
  Primer No. 13
acaaaatattaacgctcagagcgacatgccgccgctg

SEQ ID NO: 14 - DNA, 37 bp, Artificial Sequence; identical to SEQ ID NO: 12
  Primer No. 14

SEQ ID NO: 15 - DNA, 35 bp, Artificial Sequence
  Primer No. 15
acaaaatattaacgctcagaccatgtacatcccgc

SEQ ID NO: 16 - DNA, 1236 bp, Cuphea palustris
atggtggctgctgcagcaagttctgcatgcttccctgttccatccccaggagcctcccctaaacctgggaagttaggcaactggtcatcgagtttgagcccttccttgaagcccaagtcaatccccaatggcggatttcaggttaaggcaaatgccagtgcgcatcctaaggctaacggttctgcagtaactctaaagtctggcagcctcaacactcaggaggacactttgtcgtcgtcccctcctccccgggctttttttaaccagttgcctgattggagtatgcttctgactgcaatcacaaccgtcttcgtggcaccagagaagcggtggactatgtttgataggaaatctaagaggcctaacatgctcatggactcgtttgggttggagagagttgttcaggatgggctcgtgttcagacagagtttttcgattaggtcttatgaaatatgcgctgatcgaacagcctctatagagacggtgatgaaccacgtccaggaaacatcactcaatcaatgtaagagtataggtcttctcgatgacggctttggtcgtagtcctgagatgtgtaaaagggacctcatttgggtggttacaagaatgaagataatggtgaatcgctatccaacttggggcgatactatcgaggtcagtacctggctctctcaatcggggaaaatcggtatgggtcgcgattggctaataagtgattgcaacacaggagaaattcttgtaagagcaacgagtgtgtatgccatgatgaatcaaaagacgagaagattctcaaaactcccacacgaggttcgccaggaatttgcgcctcattttctggactctcctcctgccattgaagacaacgacggtaaattgcagaagtttgatgtgaagactggtgattccattcgcaagggtctaactccggggtggtatgacttggatgtcaatcagcacgtaagcaacgtgaagtacattgggtggattctcgagagtatgccaacagaagttttggagactcaggagctatgttctctcacccttgaatataggcgggaatgcggaagggacagtgtgctggagtccgtgacctctatggatccctcaaaagttggagaccggtttcagtaccggcaccttctgcggcttgaggatggggctgatatcatgaagggaagaactgagtggcggccgaagaatgcaggaactaacggggcgatatcaacaggaaagacttga

SEQ ID NO: 17 - protein, 411 aa, Cuphea palustris
MVAAAASSACFPVPSPGASPKPGKLGNWSSSLSPSLKPKSIPNGGFQVKANASAHPKANGSAVTLKSGSLNTQEDTLSSSPPPRAFFNQLPDWSMLLTAITTVFVAPEKRWTMFDRKSKRPNMLMDSFGLERVVQDGLVFRQSFSIRSYEICADRTASIETVMNHVQETSLNQCKSIGLLDDGFGRSPEMCKRDLIWVVTRMKIMVNRYPTWGDTIEVSTWLSQSGKIGMGRDWLISDCNTGEILVRATSVYAMMNQKTRRFSKLPHEVRQEFAPHFLDSPPAIEDNDGKLQKFDVKTGDSIRKGLTPGWYDLDVNQHVSNVKYIGWILESMPTEVLETQELCSLTLEYRRECGRDSVLESVTSMDPSKVGDRFQYRHLLRLEDGADIMKGRTEWRPKNAGTNGAISTGKT